(19) United States
(12) Patent Application Publication
(10) Pub. No.: US 2024/0335361 A1
(43) Pub. Date: Oct. 10, 2024

(54) MICROCAPSULES HAVING A MINERAL LAYER

(71) Applicant: Firmenich SA, Satigny (CH)

(72) Inventors: Nicholas IMPELLIZZERI, Plainsboro, NJ (US); Huda JERRI, Plainsboro, NJ (US)

(21) Appl. No.: 18/290,873

(22) PCT Filed: Jul. 20, 2022
(86) PCT No.: PCT/EP22/70307

§ 371 Date: Jan. 22, 2024

Related U.S. Application Data
(60) Provisional application No. 63/227,056, filed on Jul. 29, 2021.

(30) Foreign Application Priority Data
Sep. 7, 2021 (EP) 21195226.2

(51) Int. Cl. A61K 8/11; A61K 8/19; A61K 8/23; A61K 8/24; A61Q 5/02; A61Q 5/12; A61Q 11/00; A61Q 13/00; A61Q 15/00; A61Q 19/10; B01J 13/08; B01J 13/22; C11D 3/00; C11D 3/50

(52) U.S. Cl. CPC A61K 8/11; A61K 8/19; A61K 8/23; A61K 8/24; A61Q 5/02; A61Q 5/12; A61Q 11/00; A61Q 13/00; A61Q 15/00; A61Q 19/10; B01J 13/08; B01J 13/22; C11D 3/0015; C11D 3/505; A61K 2800/56

(57) ABSTRACT

Described herein are microcapsules including a hydrophobic material-based core, preferably a perfume or a flavour, a shell and a mineral layer onto the shell. Also described herein is a process for the preparation of the microcapsules. Also described herein are perfuming compositions and consumer products including the microcapsules, in particular perfumed consumer products in the form of fine fragrance, home care or personal care products.

### TECHNICAL FIELD

The present invention relates to the field of delivery systems. More specifically, the present invention relates to microcapsules comprising a hydrophobic ingredient-based core, preferably a perfume or a flavour, a shell and a mineral layer onto the shell. A process for the preparation of said microcapsules is also an object of the invention. Perfuming compositions and consumer products comprising said microcapsules, in particular perfumed consumer products in the form of fine fragrance, home care or personal care products, are also part of the invention.

### BACKGROUND OF THE INVENTION

One of the problems faced by the perfumery industry lies in the relatively rapid loss of the olfactive benefit provided by odoriferous compounds due to their volatility, particularly that of “top-notes”. This problem is generally tackled using a delivery system, e.g. capsules containing a perfume, to release the fragrance in a controlled manner.

In order to be successfully used in consumer products, perfume delivery systems must meet a certain number of criteria. The first requirement concerns stability in aggressive medium. In fact, delivery systems may suffer from stability problems, in particular when incorporated into surfactant-based products such as detergents, wherein said systems tend to degrade and lose efficiency in the perfume-retention ability. It is also difficult to have a good stability and a good dispersion of the capsules altogether. The dispersion factor is very important because the aggregation of capsules increases the tendency of the capsule-containing product to phase separate, which represents a real disadvantage. On the other hand, perfume delivery systems must also perform during the actual use of the end-product by the consumer, in particular in terms of odor performance, as the perfume needs to be released when required. Another issue faced for example by the perfumery industry is to provide delivery systems that are well deposited on the substrate for the treatment of which the end product is intended to be used, such as textile, skin, hair or other surfaces, so as to possibly remain on the substrate even after a rinsing step. To address this specific problem, the use of cationic capsules has been described in the prior art. Cationic capsules are also known to be better dispersed in several applications.

For example, WO 01/41915 discloses a process for the preparation of capsules carrying cationic charges. Such a process is allegedly applicable to a large variety of microcapsules, in particular polyurethane-polyurea microcapsules are mentioned. After their formation, the capsules are placed in a medium which is favourable for the treatment with cationic polymers. The treatment with cationic polymers is carried out after purification of the basic capsule slurry, in order to eliminate anionic or neutral polymers which were not incorporated in the capsule wall during formation thereof, and other free electrically charged compounds involved in the encapsulation process. In particular, the capsules are diluted, isolated and then re-suspended in water, or even washed to further eliminate anionic compounds. After the purification step, the capsules are agitated vigorously and the cationic polymers are added. Partially quaternized copolymers of polyvinylpyrrolidones are cited to this purpose, among many other suitable polymers. The described process comprises several steps following the capsule formation, said process being therefore time consuming and not economically profitable.

US 2006/0216509 also discloses a process to render polyurea capsules positively-charged. This process involves the addition, during the wall formation, of polyamines, the capsules thus bearing latent charges, depending on the pH of the medium. Once formed, the capsules are subsequently cationized by acid action or alkylation to bear permanent positive charges. The cationic compounds therefore react with the capsule wall, chemically changing the latter.

WO2009/153695 discloses a simplified process for the preparation of polyurea microcapsules bearing permanent positive charges based on the use of a specific stabilizer and which present good deposition on a substrate.

Furthermore, in addition to the improved deposition, it would be interesting having a coating which survives the wide pH ranges of different consumer application.

Despite those prior disclosures, there is still a need to improve the ability of hydrophobic ingredient (for example perfume) delivery systems to deposit on a substrate and to adhere on the substrate for leave-on and rinse-off applications, while performing in terms of hydrophobic ingredient release and stability.

The microcapsules of the invention solve this problem as they proved to show improvement in terms of deposition properties compared to what was known heretofore. Furthermore, the mineral coating proved to show stability in different type of consumer products.

### SUMMARY OF THE INVENTION

The present invention provides microcapsules with good performance in different consumer products. In particular, the growth of a specific mineral layer onto a terminating charged surface of the microcapsule provides improved deposition on different substrates. Furthermore, it has been shown that the mineral layer is stable in consumer product having different pH.

A first object of the invention is therefore a mineralized core-shell microcapsule comprising:
a) a core, preferably an oil-based core, comprising a hydrophobic ingredient, preferably a perfume;
b) a shell having a terminating charged functional surface; and
c) a mineral layer on the terminating charged functional surface, characterized in that the mineral layer comprises at least one salt chosen from the group consisting of barium salt, strontium salt, magnesium salt, and mixtures thereof.

Another object of the invention is a mineralized core-shell microcapsule slurry comprising at least one microcapsule as defined above.

A second object of the invention is a process for preparing a mineralized core-shell microcapsule slurry as defined above comprising the steps of:
(i) Preparing a core-shell microcapsule slurry comprising microcapsules having a terminating charged functional surface;
(ii) Adsorption of at least one mineral precursor on the charged surface;
(iii) Applying conditions suitable to induce crystal growth of the mineral on the charged surface to form a mineral layer,
wherein the mineral precursor is adsorbed on the charged surface by incubating the core-shell microcapsule slurry obtained in step (i) in at least one mineral precursor solution, wherein the mineral precursor solution is chosen in the group consisting of barium salt solution, strontium salt solution, magnesium salt solution, phosphate-based salt solution, sulfate-based salt solution, carbonate-based salt solution and mixtures thereof.

A third object of the invention is a perfuming composition comprising the microcapsules as defined above, wherein the oil-based core comprises a perfume.

A fourth object of the invention is a consumer product (perfumed consumer product or flavoured consumer product) comprising the microcapsules.

### BRIEF DESCRIPTION OF THE FIGURES

FIG. 1a represents scanning electron micrograph of mineralized microcapsules according to the invention (Capsules A1).

FIG. 1b represents scanning electron micrograph of mineralized microcapsules according to the invention (Capsules A2).

FIG. 2 represents scanning electron micrograph of mineralized microcapsules according to the invention (Capsules B).

FIG. 3 represents scanning electron micrograph of mineralized microcapsules according to the invention (Capsules C).

FIG. 4 represents scanning electron micrograph of smooth control microcapsules (Capsules X).

FIG. 5 represents the percentage of microcapsule deposition of mineralized microcapsules according to the invention (Capsules A1) compared to smooth control capsules (Capsules X) onto hair from a model surfactant mixture.

FIG. 6 represents the percentage of microcapsule deposition of mineralized microcapsules according to the invention (Capsules B) compared to smooth control capsules (Capsules X) onto hair from a model surfactant mixture.

FIG. 7 represents the percentage of microcapsule deposition of mineralized microcapsules according to the invention (Capsules A1) compared to smooth control capsules (Capsules X) onto cotton towel swatches from a fabric softener base.

FIG. 8 represents the percentage of microcapsule deposition of mineralized microcapsules according to the invention (Capsules A1) compared to smooth control capsules (Capsules X) onto cotton towel swatches from a detergent base.

FIG. 9 represents the stability of the mineral shell of mineralized microcapsule according in the invention (Capsules A1) after incubation in low pH fabric softener base for one month at 37° C.

### DETAILED DESCRIPTION OF THE INVENTION

Unless stated otherwise, percentages (%) are meant to designate a percentage by weight of a composition.

### Definitions

A “core-shell microcapsule”, or the similar, in the present invention is meant to designate a capsule that has a particle size distribution in the micron range (e.g. a mean diameter (d (v, 0.5)) comprised between about 1 and 3000 microns, preferably between 1 and 500 microns) and comprises an external solid oligomer-based shell or a polymeric shell and an internal continuous phase enclosed by the external shell. For avoidance of doubts coacervates are considered as core-shell microcapsules in the present invention.

By “microcapsule slurry”, it is meant microcapsule(s) that is (are) dispersed in a liquid. According to an embodiment, the slurry is an aqueous slurry, i.e the microcapsule(s) is (are) dispersed in an aqueous phase.

By “mineralized core-shell microcapsule”, it should be understood a microcapsule having a mineralized surface induced by growth of inorganic solid crystalline or amorphous inorganic material.

By “charged emulsifier” it should be understood a compound having emulsifying properties and that is negatively charged and/or positively charged. The charged emulsifier can be a charged biopolymer.

By “charged biopolymer” it should be understood a biopolymer that is negatively charged (anionic biopolymer), and/or positively charged (cationic or protonated biopolymer), and/or zwitterionic. As non-limiting examples, one may cite gum acacia, pectin, sericin, sodium caseinate and amphiphilic proteins such as soy protein, rice protein, whey protein, white egg albumin, casein, sodium caseinate, gelatin, bovine serum albumin, hydrolyzed soy protein, hydrolyzed sericin, pseudocollagen, silk protein, sericin powder, gelatin and mixtures thereof as anionic biopolymers.

By “biopolymers” it is meant biomacromolecules produced by living organisms. Biopolymers are characterized by molecular weight distributions ranging from 1,000 (1 thousand) to 1,000,000,000 (1 billion) Daltons. These macromolecules may be carbohydrates (sugar based) or proteins (amino-acid based) or a combination of both (gums) and can be linear or branched. The biopolymers according to the invention may be further chemically modified.

According to an embodiment, biopolymers are amphiphilic or anionic namely negatively charged in water at a pH greater than 9.

In the context of the invention, a “mineral layer” is composed of a stable inorganic crystalline or amorphous phase that grows normal to the terminating charged surface of the shell to yield preferably a rough, spinulose, rugose, platy, ridged or otherwise highly textured mineral aspect.

By “mineral precursor”, it should be understood a mineral precursor required for growth of the desired crystalline phase. The mineral precursor is preferably a mineral water-soluble salt containing the necessary ions for growth of the desired solid crystalline phase.

The terminology of “incubating” is used in the context of the present invention to describe the act of submerging the microcapsules in the precursor solution and allowing it time to interact with the microcapsules.

By “polyurea-based” wall or shell, it is meant that the polymer comprises urea linkages produced by either an amino-functional crosslinker or hydrolysis of isocyanate groups to produce amino groups capable of further reacting with isocyanate groups during interfacial polymerization.

By “polyurethane-based” wall or shell, it is meant that the polymer comprises urethane linkages produced by reaction of a polyol with the isocyanate groups during interfacial polymerization.

By “polyamide-based microcapsules”, it means that the microcapsule's shell comprises a polyamide material made from the reaction between an acyl chloride and at least one amino-compound. The wording “polyamide-based microcapsules” can also encompass a shell made of a composite comprising a polyamide material and another material, for example a polymer (like a protein).

For the sake of clarity, by the expression “dispersion” in the present invention it is meant a system in which particles are dispersed in a continuous phase of a different composition and it specifically includes a suspension or an emulsion.

### Core-Shell Microcapsules

A first object of the invention is therefore a mineralized core-shell microcapsule comprising:
a) a core, preferably an oil-based core, comprising a hydrophobic material, preferably a perfume;
b) a shell having a terminating charged functional surface; and
c) a mineral layer on the terminating charged functional surface,
characterized in that the mineral layer comprises at least one salt chosen from the group consisting of barium salt, strontium salt, magnesium salt, and mixtures thereof.

According to an embodiment, the mineral layer does not comprise a calcium salt.

### Hydrophobic Material

The hydrophobic material according to the invention can be “inert” material like solvents or active ingredients. The core is preferably an oil-based core.

When the hydrophobic material is an active ingredient, it is preferably chosen from the group consisting of flavors, flavor ingredients, perfumes, perfume ingredients, nutraceuticals, cosmetics, pest control agents, biocide actives and mixtures thereof.

According to a particular embodiment, the hydrophobic material comprises a mixture of a perfume with another ingredient selected from the group consisting of nutraceuticals, cosmetics, pest control agents and biocide actives.

According to an embodiment, the hydrophobic material comprises a phase change material (PCM).

According to a particular embodiment, the hydrophobic material comprises a mixture of biocide actives with another ingredient selected from the group consisting of perfumes, nutraceuticals, cosmetics, pest control agents.

According to a particular embodiment, the hydrophobic material comprises a mixture of pest control agents with another ingredient selected from the group consisting of perfumes, nutraceuticals, cosmetics, biocide actives.

According to a particular embodiment, the hydrophobic material comprises a perfume.

According to a particular embodiment, the hydrophobic material consists of a perfume.

According to a particular embodiment, the hydrophobic material consists of biocide actives.

According to a particular embodiment, the hydrophobic material consists of pest control agents.

By “perfume” (or also “perfume oil”) what is meant here is an ingredient or a composition that is preferably a liquid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odor. In other words such an ingredient, to be considered as being a perfuming one, must be recognized by a person skilled in the art as being able to at least impart or modify in a positive or pleasant way the odor of a composition, and not just as having an odor. For the purpose of the present invention, perfume oil also includes a combination of perfuming ingredients with substances which together improve, enhance or modify the delivery of the perfuming ingredients, such as perfume precursors, modulators, emulsions or dispersions, as well as combinations which impart an additional benefit beyond that of modifying or imparting an odor, such as long-lastingness, blooming, malodor counteraction, antimicrobial effect, microbial stability, pest control.

The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulfurous heterocyclic compounds and essential oils (for example Thyme oil), and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery.

In particular one may cite perfuming ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0^{2.7}]undecan-4-one, 1-methoxy-3-hexanethiol, 2-ethyl-4,4-dimethyl-1,3-oxathiane, 2,2,7/8,9/10-tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4 (8)-p-menthadiene;
Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4 (2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 3-(3,3/1,1-dimethyl-5-indanyl) propanal, 2,5-dimethyl-2-indanmethanol, 2,6,6-trimethyl-3-cyclohexene-1-carboxylate, 3-(4,4-dimethyl-1-cyclohexen-1-yl) propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl(S)-2-(1,1-dimethylpropoxy) propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, diethyl 1,4-cyclohexanedicarboxylate, 3-methyl-2-hexen-1-yl acetate, 1-[3,3-dimethylcyclohexyl]ethyl[3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{(1S)-1-[(1R)-3,3-dimethylcyclohexyl]ethoxy}-2-oxoethyl propionate, 3-methyl-5-cyclopentadecen-1-4,6,6,7,8,8-hexamethyl-1,3,4,6,7,8-hexahydrocyclopenta[g]isochromene, one, (1S,1′R)-2-[1-(3′,3′-dimethyl-1′-cyclohexyl)ethoxy]-2-methylpropyl propanoate, oxacyclohexadecan-2-one and/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.0^{2.7}]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl) ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3 (4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonane and/or 3-(3-isopropyl-1-phenyl) butanal.

It is also understood that said ingredients may also be compounds known to release in a controlled manner various types of perfuming compounds also known as properfume or profragrance. Non-limiting examples of suitable properfumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, 3-(dodecylthio)-1-(2,6,6-trimethyl-3-cyclohexen-1-yl)-1-butanone, 2-(dodecylthio) octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl)succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy) undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl) naphthalene, (2-phenethoxyvinyl)benzene, 2-(1-((3,7-dimethyloct-6-en-1-yl)oxy) prop-1-en-2-yl) naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, triethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

Preferred perfuming ingredients are those having a high steric hindrance (bulky materials) and in particular those from one of the following groups:
Group 1: perfuming ingredients comprising a cyclohexane, cyclohexene, cyclohexanone or cyclohexenone ring substituted with at least one linear or branched C₁ to C₄ alkyl or alkenyl substituent;
Group 2: perfuming ingredients comprising a cyclopentane, cyclopentene, cyclopentanone or cyclopentenone ring substituted with at least one linear or branched C₄ to C₈ alkyl or alkenyl substituent;
Group 3: perfuming ingredients comprising a phenyl ring or perfuming ingredients comprising a cyclohexane, cyclohexene, cyclohexanone or cyclohexenone ring substituted with at least one linear or branched C₅ to C₈ alkyl or alkenyl substituent or with at least one phenyl substituent and optionally one or more linear or branched C₁ to C₃ alkyl or alkenyl substituents;
Group 4: perfuming ingredients comprising at least two fused or linked C₅ and/or C₆ rings;
Group 5: perfuming ingredients comprising a camphor-like ring structure;
Group 6: perfuming ingredients comprising at least one C₇ to C₂₀ ring structure;
Group 7: perfuming ingredients having a logP value above 3.5 and comprising at least one tert-butyl or at least one trichloromethyl substitutent;

Examples of ingredients from each of these groups are:
Group 1: 2,4-dimethyl-3-cyclohexene-1-carbaldehyde (origin: Firmenich SA, Geneva, Switzerland), isocyclocitral, menthone, isomenthone, methyl 2,2-dimethyl-6-methylene-1-cyclohexanecarboxylate (origin: Firmenich SA, Geneva, Switzerland), nerone, terpineol, dihydroterpineol, terpenyl acetate, dihydroterpenyl acetate, dipentene, eucalyptol, hexylate, rose oxide, (S)-1,8-p-menthadiene-7-ol (origin: Firmenich SA, Geneva, Switzerland), 1-p-menthene-4-ol, (1RS,3RS,4SR)-3-p-mentanyl acetate, (1R,2S,4R)-4,6,6-trimethyl-bicyclo[3,1,1]heptan-2-ol, tetrahydro-4-methyl-2-phenyl-2H-pyran (origin: Firmenich SA, Geneva, Switzerland), cyclohexyl acetate, cyclanol acetate, 1,4-cyclohexane diethyldicarboxylate (origin: Firmenich SA, Geneva, Switzerland), (3RS,3aRS,6SR,7ASR)-perhydro-3,6-dimethyl-benzo[B]furan-2-one (origin: Firmenich SA, Geneva, Switzerland), ((6R)-perhydro-3,6-dimethyl-benzo[B]furan-2-one (origin: Firmenich SA, Geneva, Switzerland), 2,4,6-trimethyl-4-phenyl-1,3-dioxane, 2,4,6-trimethyl-3-cyclohexene-1-carbaldehyde;
Group 2: (E)-3-methyl-5-(2,2,3-trimethyl-3-cyclopenten-1-yl)-4-penten-2-ol (origin: Givaudan SA, Vernier, Switzerland), (1′R,E)-2-ethyl-4-(2′,2′,3′-trimethyl-3′-cyclopenten-1′-yl)-2-buten-1-ol (origin: Firmenich SA, Geneva, Switzerland), (1′R,E)-3,3-dimethyl-5-(2′,2′,3′-trimethyl-3′-cyclopenten-1′-yl)-4-penten-2-ol (origin: Firmenich SA, Geneva, Switzerland), 2-heptylcyclopentanone, methyl-cis-3-oxo-2-pentyl-1-cyclopentane acetate (origin: Firmenich SA, Geneva, Switzerland), 2,2,5-trimethyl-5-pentyl-1-cyclopentanone (origin: Firmenich SA, Geneva, Switzerland), 3,3-dimethyl-5-(2,2,3-trimethyl-3-cyclopenten-1-yl)-4-penten-2-ol (origin: Firmenich SA, Geneva, Switzerland), 3-methyl-5-(2,2,3-trimethyl-3-cyclopenten-1-yl)-2-pentanol (origin, Givaudan SA, Vernier, Switzerland);
Group 3: damascones, 1-(5,5-dimethyl-1-cyclohexen-1-yl)-4-penten-1-one (origin: Firmenich SA, Geneva, Switzerland), (1′R)-2-[2-(4′-methyl-3′-cyclohexen-1′-yl)propyl]cyclopentanone, alpha-ionone, beta-ionone, damascenone, mixture of 1-(5,5-dimethyl-1-cyclohexen-1-yl)-4-penten-1-one and 1-(3,3-dimethyl-1-cyclohexen-1-yl)-4-penten-1-one (origin: Firmenich SA, Geneva, Switzerland), 1-(2,6,6-trimethyl-1-cyclohexen-1-yl)-2-buten-1-one (origin: Firmenich SA, Geneva, Switzerland), (1S,1′R)-[1-(3′,3′-Dimethyl-1′-cyclohexyl)ethoxycarbonyl]methyl propanoate (origin: Firmenich SA, Geneva, Switzerland), 2-tert-butyl-1-cyclohexyl acetate (origin: International Flavors and Fragrances, USA), 1-(2,2,3,6-tetramethyl-cyclohexyl)-3-hexanol (origin: Firmenich SA, Geneva, Switzerland), trans-1-(2,2,6-trimethyl-1-cyclohexyl)-3-hexanol (origin: Firmenich SA, Geneva, Switzerland), (E)-3-methyl-4-(2,6,6-trimethyl-2-cyclohexen-1-yl)-3-buten-2-one, terpenyl isobutyrate, 4-(1,1-dimethylethyl)-1-cyclohexyl acetate (origin: Firmenich SA, Geneva, Switzerland), 8-methoxy-1-p-menthene, (1S,1′R)-2-[1-(3′,3′-dimethyl-1′-cyclohexyl)ethoxy]-2-methylpropyl propanoate (origin: Firmenich SA, Geneva, Switzerland), para tert-butylcyclohexanone, menthenethiol, 1-methyl-4-(4-methyl-3-pentenyl)-3-cyclohexene-1-carbaldehyde, allyl cyclohexylpropionate, cyclohexyl salicylate, 2-methoxy-4-methylphenyl methyl carbonate, ethyl 2-methoxy-4-methylphenyl carbonate, 4-ethyl-2-methoxyphenyl methyl carbonate;
Group 4: Methyl cedryl ketone (origin: International Flavors and Fragrances, USA), a mixture of (1RS,2SR,6RS,7RS,8SR)-tricyclo[5.2.1.0^{2.6}]dec-3-en-8-yl 2-methylpropanoate and (1RS,2SR,6RS,7RS,8SR)-tricyclo[5.2.1.0^{2.6}]dec-4-en-8-yl 2-methylpropanoate, vetyverol, vetyverone, 1-(octahydro-2,3,8,8-tetramethyl-2-naphtalenyl)-1-ethanone (origin: International Flavors and Fragrances, USA), (5RS,9RS,10SR)-2,6,9,10-tetramethyl-1-oxaspiro[4.5]deca-3,6-diene and the (5RS,9SR,10RS) isomer, 6-ethyl-2,10,10-trimethyl-1-oxaspiro[4.5]deca-3,6-diene, 1,2,3,5,6,7-hexahydro-1,1,2,3,3-pentamethyl-4-indenone (origin: International Flavors and Fragrances, USA), a mixture of 3-(3,3-dimethyl-5-indanyl) propanal and 3-(1,1-dimethyl-5-indanyl) propanal (origin: Firmenich SA, Geneva, Switzerland), 3′,4-dimethyl-tricyclo[6.2.1.0 (2,7)]undec-4-ene-9-spiro-2′-oxirane (origin: Firmenich SA, Geneva, Switzerland), 9/10-ethyldiene-3-oxatricyclo[6.2.1.0 (2,7)]undecane, (perhydro-5,5,8A-trimethyl-2-naphthalenyl acetate (origin: Firmenich SA, Geneva, Switzerland), octalynol, (dodecahydro-3a,6,6,9a-tetramethyl-naphtho[2,1-b]furan, origin: Firmenich SA, Geneva, Switzerland), tricyclo[5.2.1.0 (2,6)]dec-3-en-8-yl acetate and tricyclo[5.2.1.0 (2,6)]dec-4-en-8-yl acetate as well as tricyclo[5.2.1.0 (2,6)]dec-3-en-8-yl propanoate and tricyclo[5.2.1.0 (2,6)]dec-4-en-8-yl propanoate, (+)-(1S,2S,3S)-2,6,6-trimethyl-bicyclo[3.1.1]heptane-3-spiro-2′-cyclohexen-4′-one;
Group 5: camphor, borneol, isobornyl acetate, 8-isopropyl-6-methyl-bicyclo[2.2.2]oct-5-ene-2-carbaldehyde, pinene, camphene, 8-methoxycedrane, (8-methoxy-2,6,6,8-tetramethyl-tricyclo[5.3.1.0 (1,5)]undecane (origin: Firmenich SA, Geneva, Switzerland), cedrene, cedrenol, cedrol, mixture of 9-ethylidene-3-oxatricyclo[6.2.1.0 (2,7)]undecan-4-one and 10-ethylidene-3-oxatricyclo[6.2.1.0^{2.7}]undecan-4-one (origin: Firmenich SA, Geneva, Switzerland), 3-methoxy-7,7-dimethyl-10-methylene-bicyclo[4.3.1]decane (origin: Firmenich SA, Geneva, Switzerland);
Group 6: (trimethyl-13-oxabicyclo-[10.1.0]-trideca-4,8-diene (origin: Firmenich SA, Geneva, Switzerland), 9-hexadecen-16-olide (origin: Firmenich SA, Geneva, Switzerland), pentadecenolide (origin: Firmenich SA, Geneva, Switzerland), 3-methyl-(4/5)-cyclopentadecenone, (origin: SA, Firmenich Geneva, Switzerland), 3-methylcyclopentadecanone (origin: Firmenich SA, Geneva, Switzerland), pentadecanolide (origin: Firmenich SA, Geneva, Switzerland), cyclopentadecanone (origin: Firmenich SA, Geneva, Switzerland), 1-ethoxyethoxy)cyclododecane (origin: Firmenich SA, Geneva, Switzerland), 1,4-dioxacycloheptadecane-5,17-dione, 4,8-cyclododecadien-1-one;
Group 7: (+−)-2-methyl-3-[4-(2-methyl-2-propanyl)phenyl]propanal (origin: Givaudan SA, Vernier, Switzerland), 2,2,2-trichloro-1-phenylethyl acetate.

Preferably, the perfume comprises at least 30%, preferably at least 50%, more preferably at least 60% of ingredients selected from Groups 1 to 7, as defined above. More preferably said perfume comprises at least 30%, preferably at least 50% of ingredients from Groups 3 to 7, as defined above. Most preferably said perfume comprises at least 30%, preferably at least 50% of ingredients from Groups 3, 4, 6 or 7, as defined above.

According to another preferred embodiment, the perfume comprises at least 30%, preferably at least 50%, more preferably at least 60% of ingredients having a logP above 3, preferably above 3.5 and even more preferably above 3.75.

According to a particular embodiment, the perfume used in the invention contains less than 10% of its own weight of primary alcohols, less than 15% of its own weight of secondary alcohols and less than 20% of its own weight of tertiary alcohols. Advantageously, the perfume used in the invention does not contain any primary alcohols and contains less than 15% of secondary and tertiary alcohols.

According to an embodiment, the oil phase (or the oil-based core) comprises:
25-100 wt %, preferably 25-98% of a perfume oil comprising at least 15 wt % of high impact perfume raw materials having a Log T<−4, and
0-75 wt %, preferably 2-75% of a density balancing material having a density greater than 1.07 g/cm³.

“High impact perfume raw materials” should be understood as perfume raw materials having a LogT<−4. The odor threshold concentration of a chemical compound is determined in part by its shape, polarity, partial charges and molecular mass. For convenience, the odor threshold concentration is presented as the common logarithm of the threshold concentration, i.e., Log [Threshold] (“LogT”).

A “density balancing material” should be understood as a material having a density greater than 1.07 g/cm³ and having preferably low or no odor.

The density of a component is defined as the ratio between its mass and its volume (g/cm³).

Several methods are available to determine the density of a component.

One may refer for example to the ISO 298:1998 method to measure d20 densities of essential oils.

According to an embodiment, the density balancing material is chosen in the group consisting of benzyl salicylate, benzyl benzoate, cyclohexyl salicylate, benzyl phenylacetate, phenylethyl phenoxyacetate, triacetin, methyl and ethyl salicylate, benzyl cinnamate, and mixtures thereof.

According to a particular embodiment, the density balancing material is chosen in the group consisting of benzyl salicylate, benzyl benzoate, cyclohexyl salicylate and mixtures thereof.

The odor threshold concentration of a perfuming compound is determined by using a gas chromatograph (“GC”). Specifically, the gas chromatograph is calibrated to determine the exact volume of the perfume oil ingredient injected by the syringe, the precise split ratio, and the hydrocarbon response using a hydrocarbon standard of known concentration and chain-length distribution. The air flow rate is accurately measured and, assuming the duration of a human inhalation to last 12 seconds, the sampled volume is calculated. Since the precise concentration at the detector at any point in time is known, the mass per volume inhaled is known and hence the concentration of the perfuming compound. To determine the threshold concentration, solutions are delivered to the sniff port at the back-calculated concentration. A panelist sniffs the GC effluent and identifies the retention time when odor is noticed. The average across all panelists determines the odor threshold concentration of the perfuming compound. The determination of odor threshold is described in more detail in C. Vuilleumier et al., Multidimensional Visualization of Physical and Perceptual Data Leading to a Creative Approach in Fragrance Development, Perfume & Flavorist, Vol. 33, September 2008, pages 54-61.

The nature of high impact perfume raw materials having a Log T<−4 and density balancing material having a density greater than 1.07 g/cm³ are described in WO2018115250, the content of which are included by reference.

According to an embodiment, the high impact perfume raw materials having a Log T<−4 are selected from the group consisting of (+−)-1-methoxy-3-hexanethiol, 4-(4-hydroxy-1-phenyl)-2-butanone, 2-methoxy-4-(1-propenyl)-1-phenyl acetate, pyrazobutyle, 3-propylphenol, 1-(3-methyl-1-benzofuran-2-yl) ethanone, 2-(3-phenylpropyl)pyridine, 1-(3,3/5,5-dimethyl-1-cyclohexen-1-yl)-4-penten-1-one, 1-(5,5-dimethyl-1-cyclohexen-1-yl)-4-penten-1-one, a mixture comprising (3RS,3aRS,6SR,7ASR)-perhydro-3,6-dimethyl-benzo[b]furan-2-one and (3SR,3aRS,6SR,7ASR)-perhydro-3,6-dimethyl-benzo[b]furan-2-one, (+−)-1-(5-ethyl-5-methyl-1-cyclohexen-1-yl)-4-penten-1-one, (1'S,3′R)-1-methyl-2-[(1′,2′,2′-trimethylbicyclo[3.1.0]hex-3′-yl)methyl]cyclopropyl}methanol, (+−)-3-mercaptohexyl acetate, (2E)-1-(2,6,6-trimethyl-1,3-cyclohexadien-1-yl)-2-buten-1-one, H-methyl-2h-1,5-benzodioxepin-3 (4H)-one, (2E,6Z)-2,6-nonadien-1-ol, (4Z)-4-dodecenal, (+−)-4-hydroxy-2,5-dimethyl-3 (2H)-furanone, methyl 2,4-dihydroxy-3,6-dimethylbenzoate, 3-methylindole, (+−)-perhydro-4alpha,8abeta-dimethyl-4a-naphthalenol, patchoulol, 2-methoxy-4-(1-propenyl) phenol, mixture comprising (+−)-5,6-dihydro-4-methyl-2-phenyl-2H-pyran and tetrahydro-4-methylene-2-phenyl-2H-pyran, mixture comprising 4-methylene-2-phenyltetrahydro-2H-pyran and (+−)-4-methyl-2-phenyl-3,6-dihydro-2H-pyran, 4-hydroxy-3-methoxybenzaldehyde, nonylenic aldehyde, 2-methoxy-4-propylphenol, 3-methyl-5-phenyl-2-pentenenitrile, 1-(spiro [4.5]dec-6/7-en-7-yl)-4-penten-1-one, 2-methoxynaphthalene, (−)-(3aR,5AS,9AS,9BR)-3a,6,6,9a-tetramethyldodecahydronaphtho[2,1-b]furan, 5-nonanolide, (3aR,5AS,9AS,9BR)-3a,6,6,9a-tetramethyldodecahydronaphtho[2,1-b]furan, 7-isopropyl-2H,4H-1,5-benzodioxepin-3-one, coumarin, 4-methylphenyl isobutyrate, (2E)-1-(2,6,6-trimethyl-1,3-cyclohexadien-1-yl)-2-buten-1-one, beta,2,2,3-tetramethyl-delta-methylene-3-cyclopentene-1-butanol, delta damascone ((2E)-1-[(1RS,2SR)-2,6,6-trimethyl-3-cyclohexen-1-yl]-2-buten-1-one), (+−)-3,6-dihydro-4,6-dimethyl-2-phenyl-2h-pyran, anisaldehyde, paracresol, 3-ethoxy-4-hydroxybenzaldehyde, methyl 2-aminobenzoate, ethyl methylphenylglycidate, octalactone gamma, ethyl 3-phenyl-2-propenoate, (−)-(2E)-2-ethyl-4-[(1R)-2,2,3-trimethyl-3-cyclopenten-1-yl]-2-buten-1-ol, paracresyl acetate, dodecalactone, tricyclone, (+)-(3R,5Z)-3-methyl-5-cyclopentadecen-1-one, undecalactone, (1R,4R)-8-mercapto-3-p-menthanone, (3S,3AS,6R,7AR)-3,6-dimethylhexahydro-1-benzofuran-2 (3H)-one, beta ionone, (+−)-6-pentyltetrahydro-2H-pyran-2-one, (3E,5Z)-1,3,5-undecatriene, 10-undecenal, (9E)-9-undecenal (9Z)-9-undecenal, (Z)-4-decenal, (+−)-ethyl 2-methylpentanoate, 1,2-diallyldisulfane, 2-tridecenenitrile, 3-tridecenenitrile, (+−)-2-ethyl-4,4-dimethyl-1,3-oxathiane, (+)-(3R,5Z)-3-methyl-5-cyclopentadecen-1-one, 3-(4-tert-butylphenyl) propanal, allyl (cyclohexyloxy)acetate, methylnaphthylketone, (+−)-(4E)-3-methyl-4-cyclopentadecen-1-one, (+−)-5E3-methyl-5-cyclopentadecen-1-one, cyclopropylmethyl 3-hexenoate, (4E)-4-methyl-5-(4-methylphenyl)-4-pentenal, (+−)-1-(5-propyl-1,3-benzodioxol-2-yl) ethanone, 4-methyl-2-pentylpyridine, (+−)-(E)-3-methyl-4-(2,6,6-trimethyl-2-cyclohexen-1-yl)-3-buten-2-one, (3aRS,5aSR,9aSR,9bRS)-3a,6,6,9a-tetramethyldodecahydronaphtho[2,1-b]furan, (2S,5R)-5-methyl-2-(2-propanyl)cyclohexanone oxime, 6-hexyltetrahydro-2H-pyran-2-one, (+−)-3-(3-isopropyl-1-phenyl) butanal, methyl 2-(3-oxo-2-pentylcyclopentyl)acetate, 1-(2,6,6-trimethyl-1-cyclohex-2-enyl) pent-1-en-3-one, indol, 7-propyl-2H,4H-1,5-benzodioxepin-3-one, ethyl praline, (4-methylphenoxy) acetaldehyde, ethyl tricyclo[5.2.1.0.2.6]decane-2-carboxylate, (+)-(1′S,2S,E)-3,3-dimethyl-5-(2′,2′,3′-trimethyl-3′-cyclopenten-1′-yl)-4-penten-2-ol, (4E)-3,3-dimethyl-5-[(1R)-2,2,3-trimethyl-3-cyclopenten-1-yl]-4-penten-2-ol, 8-isopropyl-6-methyl-bicyclo[2.2.2]oct-5-ene-2-carbaldehyde, methylnonylacetaldehyde, 4-formyl-2-methoxyphenyl 2-methylpropanoate, (E)-4-decenal, (+−)-2-ethyl-4-(2,2,3-trimethyl-3-cyclopenten-1-yl)-2-buten-1-ol, (1R,5R)-4,7,7-trimethyl-6-thiabicyclo[3.2.1]oct-3-ene, (1R,4R,5R)-4,7,7-trimethyl-6-thiabicyclo[3.2.1]octane, (−)-(3R)-3,7-dimethyl-1,6-octadien-3-ol, (E)-3-phenyl-2-propenenitrile, 4-methoxybenzyl acetate, (E)-3-methyl-5-(2,2,3-trimethyl-3-cyclopenten-1-yl)-4-penten-2-ol, allyl (2/3-methylbutoxy)acetate, (+−)-(2E)-1-(2,6,6-trimethyl-2-cyclohexen-1-yl)-2-buten-1-one, (1E)-1-(2,6,6-trimethyl-1-cyclohexen-1-yl)-1-penten-3-one, and mixtures thereof.

According to an embodiment, perfume raw materials having a Log T<−4 are chosen in the group consisting of aldehydes, ketones, alcohols, phenols, esters lactones, ethers, epoxydes, nitriles and mixtures thereof.

According to an embodiment, perfume raw materials having a Log T<−4 comprise at least one compound chosen in the group consisting of alcohols, phenols, esters lactones, ethers, epoxydes, nitriles and mixtures thereof, preferably in amount comprised between 20 and 70% by weight based on the total weight of the perfume raw materials having a Log T<−4.

According to an embodiment, perfume raw materials having a Log T<−4 comprise between 20 and 70% by weight of aldehydes, ketones, and mixtures thereof based on the total weight of the perfume raw materials having a Log T<−4.

The remaining perfume raw materials contained in the oil-based core may have therefore a Log T>−4.

According to an embodiment, the perfume raw materials having a Log T>−4 are chosen in the group consisting of ethyl 2-methylbutyrate, (E)-3-phenyl-2-propenyl acetate, (+−)-6/8-sec-butylquinoline, (+−)-3-(1,3-benzodioxol-5-yl)-2-methylpropanal, verdyl propionate, 1-(octahydro-2,3,8,8-tetramethyl-2-naphtalenyl)-1-ethanone, methyl 2-((1RS,2RS)-3-oxo-2-pentylcyclopentyl)acetate, (+−)-(E)-4-methyl-3-decen-5-ol, 2,4-dimethyl-3-cyclohexene-1-carbaldehyde, 1,3,3-trimethyl-2-oxabicyclo[2.2.2]octane, tetrahydro-4-methyl-2-(2-methyl-1-propenyl)-2H-pyran, dodecanal, 1-oxa-12/13-cyclohexadecen-2-one, (+−)-3-(4-isopropylphenyl)-2-methylpropanal, aldehyde C11, (+−)-2,6-dimethyl-7-octen-2-ol, allyl 3-cyclohexylpropanoate, (Z)-3-hexenyl acetate, 5-methyl-2-(2-propanyl)cyclohexanone, allyl heptanoate, 2-(2-methyl-2-propanyl)cyclohexyl acetate, 1,1-dimethyl-2-phenylethyl butyrate, geranyl acetate, neryl acetate, (+−)-1-phenylethyl acetate, 1,1-dimethyl-2-phenylethyl acetate, 3-methyl-2-butenyl acetate, ethyl 3-oxobutanoate, (2Z)-ethyl 3-hydroxy-2-butenoate, 8-p-menthanol, 8-p-menthanyl acetate, 1-p-menthanyl acetate, (+−)-2-(4-methyl-3-cyclohexen-1-yl)-2-propanyl acetate, (+−)-2-methylbutyl butanoate, 2-{(1S)-1-[(1R)-3,3-dimethylcyclohexyl]ethoxy}-2-oxoethyl propionate, 3,5,6-trimethyl-3-cyclohexene-1-carbaldehyde, 2,4,6-trimethyl-3-cyclohexene-1-carbaldehyde, 2-cyclohexylethyl acetate, octanal, ethyl butanoate, (+−)-(3E)-4-(2,6,6-trimethyl-1/2-cyclohexen-1-yl)-3-buten-2-one, 1-[(1RS,6SR)-2,2,6-trimethylcyclohexyl]-3-hexanol, 1,3,3-trimethyl-2-oxabicyclo[2.2.2]octane, 1,3,3-trimethyl-2-oxabicyclo[2.2.2]octane, ethyl hexanoate, undecanal, decanal, 2-phenylethyl acetate, (1S,2S,4S)-1,7,7-trimethylbicyclo[2.2.1]heptan-2-ol, (1S,2R,4S)-1,7,7-trimethylbicyclo[2.2.1]heptan-2-ol), (+−)-3,7-dimethyl-3-octanol, 1-methyl-4-(2-propanylidene)cyclohexene, (+)-(R)-4-(2-methoxypropan-2-yl)-1-methylcyclohex-1-ene, verdyl acetate, (3R)-1-[(1R,6S)-2,2,6-trimethylcyclohexyl]-3-hexanol, (3S)-1-[(1R,6S)-2,2,6-trimethylcyclohexyl]-3-hexanol, (3R)-1-[(1S,6S)-2,2,6-trimethylcyclohexyl]-3-hexanol, (+)-(1S,1′R)-2-[1-(3′,3′-dimethyl-1′-cyclohexyl)ethoxy]-2-methylpropyl propanoate, and mixtures thereof.

According to an embodiment, the perfume formulation comprises
0 to 60 wt. % of a hydrophobic solvent (based on the total weight of the perfume formulation),
40 to 100 wt. % of a perfume oil (based on the total weight of the perfume formulation), wherein the perfume oil has at least two, preferably all of the following characteristics:
   at least 35%, preferably at least 40%, preferably at least 50%, more preferably at least 60% of perfuming ingredients having a log P above 3, preferably above 3.5,
   at least 20%, preferably at least 25%, preferably at least 30%, more preferably at least 40% of Bulky materials of groups 1 to 6, preferably 3 to 6 as defined previously and
   at least 15%, preferably at least 20%, more preferably at least 25%, even more preferably at least 30% of high impact perfume materials having a Log T<−4 as defined previously,
      optionally, further hydrophobic active ingredients.

According to a particular embodiment, the perfume comprises 0 to 60 wt. % of a hydrophobic solvent.

According to a particular embodiment, the hydrophobic solvent is a density balancing material preferably chosen in the group consisting of benzyl salicylate, benzyl benzoate, cyclohexyl salicylate, benzyl phenylacetate, phenylethyl phenylacetate, triacetin, ethyl citrate, methyl and ethyl salicylate, benzyl cinnamate, and mixtures thereof.

In a particular embodiment, the hydrophobic solvent has Hansen Solubility Parameters compatible with entrapped perfume oil.

The term “Hansen solubility parameter” is understood refers to a solubility parameter approach proposed by Charles Hansen used to predict polymer solubility and was developed around the basis that the total energy of vaporization of a liquid consists of several individual parts. To calculate the “weighted Hansen solubility parameter” one must combine the effects of (atomic) dispersion forces, (molecular) permanent dipole-permanent dipole forces, and (molecular) hydrogen bonding (electron exchange). The weighted Hansen solubility parameter” is calculated as (δD²+δP²+δH²)^{0.5}, wherein δD is the Hansen dispersion value (also referred to in the following as the atomic dispersion fore), δP is the Hansen polarizability value (also referred to in the following as the dipole moment), and δH is the Hansen Hydrogen-bonding (“h-bonding”) value (also referred to in the following as hydrogen bonding). For a more detailed description of the parameters and values, see Charles Hansen, The Three Dimensional Solubility Parameter and Solvent Diffusion Coefficient, Danish Technical Press (Copenhagen, 1967).

Euclidean difference in solubility parameter between a fragrance and a solvent is calculated as (4*(δDₛₒₗᵥₑₙₜ-δD_{fragrance})²+(δPₛₒₗᵥₑₙₜ-δP_{fragrance})²+(δHₛₒₗᵥₑₙₜ-δH_{fragrance})²)^{0.5}, in which δDₛₒₗᵥₑₙₜ, δPₛₒₗᵥₑₙₜ, and δHₛₒₗᵥₑₙₜ, are the Hansen dispersion value, Hansen polarizability value, and Hansen h-bonding values of the solvent, respectively; and δD_{fragrance}, δP_{fragrance}, and δH_{fragrance} are the Hansen dispersion value, Hansen polarizability value, and Hansen h-bonding values of the fragrance, respectively.

In a particular embodiment, the perfume oil and the hydrophobic solvent have at least two Hansen solubility parameters selected from a first group consisting of: an atomic dispersion force (δD) from 12 to 20, a dipole moment (δP) from 1 to 8, and a hydrogen bonding (δH) from 2.5 to 11.

In a particular embodiment, the perfume oil and the hydrophobic solvent have at least two Hansen solubility parameters selected from a second group consisting of: an atomic dispersion force (δD) from 12 to 20, preferably from 14 to 20, a dipole moment (δP) from 1 to 8, preferably from 1 to 7, and a hydrogen bonding (δH) from 2.5 to 11, preferably from 4 to 11.

In a particular embodiment, at least 90% of the perfume oil, preferably at least 95% of the perfume oil, most preferably at least of 98% of the perfume oil has at least two Hansen solubility parameters selected from a first group consisting of: an atomic dispersion force (δD) from 12 to 20, a dipole moment (δP) from 1 to 8, and a hydrogen bonding (δH) from 2.5 to 11.

In a particular embodiment, the perfume oil and the hydrophobic solvent have at least two Hansen solubility parameters selected from a second group consisting of: an atomic dispersion force (δD) from 12 to 20, preferably from 14 to 20, a dipole moment (δP) from 1 to 8, preferably from 1 to 7, and a hydrogen bonding (δH) from 2.5 to 11, preferably from 4 to 11.

According to an embodiment, the perfuming formulation comprises a fragrance modulator (that can be used in addition to the hydrophobic solvent when present or as substitution of the hydrophobic solvent when there is no hydrophobic solvent).

Preferably, the fragrance modulator is defined as a fragrance material with
i. a vapor pressure of less than 0.0008 Torr at 22° C.;
ii. a clogP of 3.5 and higher, preferably 4.0 and higher and more preferably 4.5
iii. at least two Hansen solubility parameters selected from a first group consisting of: an atomic dispersion force from 12 to 20, a dipole moment from 1 to 7, and a hydrogen bonding from 2.5 to 11,
iv. at least two Hansen solubility parameters selected from a second group consisting of: an atomic dispersion force from 14 to 20, a dipole moment from 1 to 8, and a hydrogen bonding from 4 to 11, when in solution with a compound having a vapor pressure range of 0.0008 to 0.08 Torr at 22° C.

Preferably, as examples the following ingredients can be listed as modulators but the list in not limited to the following materials: alcohol C12, oxacyclohexadec-12/13-en-2-one, 3-[(2′,2′,3′-trimethyl-3′-cyclopenten-1′-yl)methoxy]-2-butanol, cyclohexadecanone, (Z)-4-cyclopentadecen-1-one, cyclopentadecanone, (8Z)-oxacycloheptadec-8-en-2-one, 2-[5-(tetrahydro-5-methyl-5-vinyl-2-furyl)-tetrahydro-5-methyl-2-furyl]-2-propanol, muguet aldehyde, 1,5,8-trimethyl-13-oxabicyclo[10.1.0]trideca-4,8-diene, (+−)-4,6,6,7,8,8-hexamethyl-1,3,4,6,7,8-hexahydrocyclopenta[g]isochromene, (+)-(1S,2S,3S,5R)-2,6,6-trimethylspiro[bicyclo[3.1.1]heptane-3,1′-cyclohexane]-2′-en-4′-one, oxacyclohexadecan-2-one, 2-{(1S)-1-[(1R)-3,3-dimethylcyclohexyl]ethoxy}-2-oxoethyl propionate, (+)-(4R,4aS,6R)-4,4a-dimethyl-6-(1-propen-2-yl)-4,4a,5,6,7,8-hexahydro-2 (3H)-naphthalenone, amylcinnamic aldehyde, hexylcinnamic aldehyde, hexyl salicylate, (1E)-1-(2,6,6-trimethyl-1-cyclohexen-1-yl)-1,6-heptadien-3-one, (9Z)-9-cycloheptadecen-1-one.

According to a particular embodiment, the hydrophobic material is free of any active ingredient (such as perfume). According to this particular embodiment, it comprises, preferably consists of hydrophobic solvents, preferably chosen in the group consisting of isopropyl myristate, tryglycerides (e.g. Neobee® MCT oil, vegetable oils), D-limonene, silicone oil, mineral oil, and mixtures thereof with optionally hydrophilic solvents preferably chosen in the group consisting of 1,4-butanediol, benzyl alcohol, triethyl citrate, triacetin, benzyl acetate, ethyl acetate, propylene glycol (1,2-propanediol), 1,3-propanediol, dipropylene glycol, glycerol, glycol ethers and mixtures thereof.

The term “biocide” refers to a chemical substance capable of killing living organisms (e.g. microorganisms) or reducing or preventing their growth and/or accumulation. Biocides are commonly used in medicine, agriculture, forestry, and in industry where they prevent the fouling of, for example, water, agricultural products including seed, and oil pipelines. A biocide can be a pesticide, including a fungicide, herbicide, insecticide, algicide, molluscicide, miticide and rodenticide; and/or an antimicrobial such as a germicide, antibiotic, antibacterial, antiviral, antifungal, antiprotozoal and/or antiparasite.

As used herein, a “pest control agent” indicates a substance that serves to repel or attract pests, to decrease, inhibit or promote their growth, development or their activity. Pests refer to any living organism, whether animal, plant or fungus, which is invasive or troublesome to plants or animals, pests include insects notably arthropods, mites, spiders, fungi, weeds, bacteria and other microorganisms.

According to a particular embodiment, the hydrophobic material is free of any active ingredient (such as perfume). According to this particular embodiment, it comprises, preferably consists of hydrophobic solvents, preferably chosen in the group consisting of isopropyl myristate, tryglycerides (e.g. Neobee® MCT oil, vegetable oils), D-limonene, silicone oil, mineral oil, and mixtures thereof with optionally hydrophilic solvents preferably chosen in the group consisting of 1,4-butanediol, benzyl alcohol, triethyl citrate, triacetin, benzyl acetate, ethyl acetate, propylene glycol (1,2-propanediol), 1,3-propanediol, dipropylene glycol, glycerol, glycol ethers and mixtures thereof.

By “flavor oil”, it is meant here a flavoring ingredient or a mixture of flavoring ingredients, solvents or adjuvants of current use for the preparation of a flavoring formulation, i.e. a particular mixture of ingredients which is intended to be added to an edible composition or chewable product to impart, improve or modify its organoleptic properties, in particular its flavor and/or taste. Flavoring ingredients are well known to a person skilled in the art and their nature does not warrant a detailed description here, which in any case would not be exhaustive, the skilled flavorist being able to select them on the basis of his general knowledge and according to the intended use or application and the organoleptic effect it is desired to achieve. Many of these flavoring ingredients are listed in reference texts such as in the book by S. Arctander, Perfume and Flavor Chemicals, 1969, Montclair, N.J., USA, or its more recent versions, or in other works of similar nature such as Fenaroli's Handbook of Flavor Ingredients, 1975, CRC Press or Synthetic Food Adjuncts, 1947, by M. B. Jacobs, van Nostrand Co., Inc. Solvents and adjuvants of current use for the preparation of a flavoring formulation are also well known in the art.

In a particular embodiment, the flavor is a mint flavor. In a more particular embodiment, the mint is selected from the group consisting of peppermint and spearmint.

In a further embodiment, the flavor is a cooling agent or mixtures thereof.

In another embodiment, the flavor is a menthol flavor.

Flavors that are derived from or based on fruits where citric acid is the predominant, naturally-occurring acid include but are not limited to, for example, citrus fruits (e.g. lemon, lime), limonene, strawberry, orange, and pineapple. In one embodiment, the flavors food is lemon, lime or orange juice extracted directly from the fruit. Further embodiments of the flavor comprise the juice or liquid extracted from oranges, lemons, grapefruits, key limes, citrons, clementines, mandarins, tangerines, and any other citrus fruit, or variation or hybrid thereof. In a particular embodiment, the flavor comprises a liquid extracted or distilled from oranges, lemons, grapefruits, key limes, citrons, clementines, mandarins, tangerines, any other citrus fruit or variation or hybrid thereof, pomegranates, kiwifruits, watermelons, apples, bananas, blueberries, melons, ginger, bell peppers, cucumbers, passion fruits, mangos, pears, tomatoes, and strawberries.

In a particular embodiment, the flavor comprises a composition that comprises limonene, in a particular embodiment, the composition is a citrus that further comprises limonene.

In another particular embodiment, the flavor comprises a flavor selected from the group comprising strawberry, orange, lime, tropical, berry mix, and pineapple.

The phrase flavor includes not only flavors that impart or modify the smell of foods but include taste imparting or modifying ingredients. The latter do not necessarily have a taste or smell themselves but are capable of modifying the taste that other ingredients provides, for instance, salt enhancing ingredients, sweetness enhancing ingredients, umami enhancing ingredients, bitterness blocking ingredients and so on.

In a further embodiment, suitable sweetening components may be included in the particles described herein. In a particular embodiment, a sweetening component is selected from the group consisting of sugar (e.g., but not limited to sucrose), a stevia component (such as but not limited to stevioside or rebaudioside A), sodium cyclamate, aspartame, sucralose, sodium saccharine, and Acesulfam K or mixtures thereof.

According an embodiment, the hydrophobic material represents between about 10% and 95% by weight, relative to the total weight of the oil phase. According another embodiment, the hydrophobic material represents between about 10% and 80% by weight, relative to the total weight of the oil phase. According another embodiment, the hydrophobic material represents between about 10% and 60% by weight, relative to the total weight of the oil phase. According another embodiment, the hydrophobic material represents between about 15% and 45% by weight, relative to the total weight of the oil phase.

According to an embodiment, the core of the microcapsule is liquid.

According to another embodiment, the core of the microcapsule is solid.

According to an embodiment, the mineral layer forms a spinulose surface covered by small spikes, ridges or platy protuberances perpendicular to the terminating charged functional surface (typically having a length between 100 and 600 nm and having an aspect ratio greater than 1).

Indeed, the surface of the mineral layer can have a rough, spiny, spiky, ridged, rugose, orthorhombic, studded, cubic, dendritic or textured appearance with rough heterogeneous crystalline features over the surface.

According to a particular embodiment, the mineral layer has an arithmetical mean roughness value (Rₐ) greater than 15 nm, preferably greater than 50 nm and/or a mean roughness depth (R_{z}) greater than 50 nm, preferably greater than 100 nm.

The instrument used in the present invention to evaluate surface features and determine surface roughness parameters Rₐ and R_{z} is a Keyence VK-X series confocal laser scanning microscope profilometer with a violet range laser. A Dimension ICON Atomic Force Microscope (AFM) from Bruker was also used to evaluate the surface features.

Roughness parameters are well known by the skilled person in the art and can be defined as follows.

The arithmetical mean roughness value (Rₐ) is the average deviation of the surface height from the mean height of the roughness profile. The mean roughness depth (R_{z}) is the mean localized maximum roughness, or average peak-to-valley height difference per unit length analyzed.

A good deposition can be achieved with the microcapsules of the invention due notably to this specific spinulose or rough textured surface that can adhere to the targeted substrates.

### Nature/Formation of the Shell

According to an embodiment, the shell is a polymeric shell.

According to another embodiment, the shell does not comprise any polymeric material.

According to an embodiment, the shell comprises hydrogel. According to another embodiment, the shell consists of hydrogel (i.e coacervate).

According to an embodiment, the polymeric shell is formed by interfacial polymerisation or by precipitation in the presence of a charged emulsifier.

One of the essential features of the present invention is that the shell, preferably a polymeric shell, has a terminating charged functional surface covered by a mineral layer. Different ways can be used to impart such charged surface on the polymeric shell. The terminating charged functional surface can be anionic or cationic.

According to a particular embodiment, the terminating charged functional surface is a terminating anionic functional surface.

### Emulsifier=Anionic Emulsifier

According to a first embodiment, the charged emulsifier is an anionic emulsifier and forms an anionic surface once the interfacial polymerization is completed.

The anionic emulsifier can be amphiphilic materials, colloidal stabilizers or biopolymers.

According to an embodiment, the anionic emulsifier is selected from the group consisting of polyvinyl alcohol, polyvinyl pyrrolidone, gum acacia, casein, sodium caseinate, soy protein, rice protein, whey protein, white egg albumin, gelatin, bovine serum albumin, hydrolyzed soy protein, hydrolyzed sericin, pseudocollagen, silk protein, sericin powder, sugar beet pectin, gelatin and mixtures thereof.

According to an embodiment, gum acacia is preferred.

According to an embodiment, whey protein and/or sodium caseinate are preferred.

According to a particular embodiment, the anionic surface (formed by the anionic emulsifier) is the terminating anionic functional surface that is directly covered by the mineral layer.

However, to improve the bonding of the mineral layer on the anionic surface, a polyelectrolyte scaffolding composed of oppositely-charge polyelectrolyte layer can be disposed between the anionic surface and the mineral layer.

Thus, according to a particular embodiment, the microcapsule comprises a polyelectrolyte scaffolding on the anionic surface, said polyelectrolyte scaffolding including at least one cationic polyelectrolyte layer and at least one anionic polyelectrolyte layer, the terminating layer being an anionic polyelectrolyte layer to form the terminating anionic functional surface of the shell.

According to this embodiment, the first layer of the polyelectrolyte scaffolding is a cationic polyelectrolyte layer disposed on the anionic surface (formed by the anionic emulsifier) and the last layer of the polyelectrolyte scaffolding is an anionic polyelectrolyte layer to form the terminating anionic functional surface on which the mineral layer is coated.

The number of layers of the polyelectrolyte scaffolding is not particularly limited.

According to a particular embodiment, the polyelectrolyte scaffolding consists of two pairs of oppositely charged polyelectrolytes layers.

It means that according to this embodiment, the microcapsule according to the invention comprises the following successive layers on the polymeric shell, a first cationic polyelectrolyte layer on the anionic surface (formed by the anionic emulsifier), a first negative polyelectrolyte layer, a second cationic polyelectrolyte layer, a second negative polyelectrolyte layer (forming the terminating anionic functional surface) and a mineral layer.

### Emulsifier=Cationic Emulsifier

According to a second embodiment,
the charged emulsifier is a cationic emulsifier that forms a cationic surface, and
the microcapsule comprises at least one anionic polyelectrolyte layer on the cationic surface.

According to an embodiment, the cationic emulsifier is obtained by mixing a weakly anionic emulsifier (such as PVOH) with a strongly charged cationic polymer or polyquaternium (such as Salcare® SC-60 by BASF).

As non-limiting examples of cationic emulsifiers, one may cite for example cationic functionalized polyvinyl alcohol (as an example, cationic C-506 by Kuraray) or chitosan at an appropriate pH (typically at a weakly acidic pH (approximately pH 6.5).

According to a particular embodiment, the anionic surface (formed by the anionic polyelectrolyte layer) is the terminating anionic functional surface that is directly covered by the mineral layer.

According to another embodiment, at least one cationic polyelectrolyte layer and at least a second anionic polyelectrolyte layer are deposited successively on the anionic polyelectrolyte layer.

However, this embodiment is not limited to only one pair of opposite polyelectrolyte layers but includes 2, 3, 4 or even more of pair of opposite polyelectrolyte layers, with the proviso that the last polyelectrolyte layer is an anionic polyelectrolyte layer to form the terminating anionic functional surface.

According to an embodiment, the cationic polyelectrolyte layer is chosen in the group consisting of poly(allylamine hydrochloride), poly-L-lysine and chitosan.

According to another embodiment, the anionic polyelectrolyte layer is chosen in the group consisting of poly(sodium 4 styrene sulfonate) (PSS), polyacrylic acid, polyethylene imine, humic acid, carrageenan, gum acacia, and mixtures thereof.

According to a particular embodiment, the anionic polyelectrolyte layer is PSS.

The nature of the polymeric shell of the microcapsules of the invention can vary. As non-limiting examples, the polymeric shell can comprise a material selected from the group consisting of polyurea, polyurethane, polyamide, polyhydroxyalkanoates, polyacrylate, polyesters, polyaminoesters, polyepoxides, polysiloxane, polycarbonate, polysulfonamide, urea formaldehyde, melamine formaldehyde resin, melamine formaldehyde resin cross-linked with polyisocyanate or aromatic polyols, melamine urea resin, melamine glyoxal resin, gelatin/gum arabic shell wall, and mixtures thereof.

According to an embodiment, the microcapsule comprises a composite shell comprising a first material and a second material, wherein the first material and the second material are different, the first material is a coacervate, the second material is a polymeric material. In a particular embodiment, the weight ratio between the first material and the second material is comprised between 50:50 and 99.9:0.1. In a particular embodiment, the coacervate comprises a first polyelectrolyte, preferably selected among proteins (such as gelatin), polypeptides or polysaccharides (such as chitosan), most preferably Gelatin and a second polyelectrolyte, preferably alginate salts, cellulose derivatives guar gum, pectinate salts, carrageenan, polyacrylic and methacrylic acid or xanthan gum, or yet plant gums such as acacia gum (Gum Arabic), most preferably Gum Arabic. The coacervate first material can be hardened chemically using a suitable cross-linker such as glutaraldehyde, glyoxal, formaldehyde, tannic acid or genipin or can be hardened enzymatically using an enzyme such as transglutaminase. The second polymeric material can be selected from the group consisting of polyurea, polyurethane, polyamide, polyester, polyacrylate, polysiloxane, polycarbonate, polysulfonamide, polymers of urea and formaldehyde, melamine and formaldehyde, melamine and urea, or melamine and glyoxal and mixtures thereof, preferably polyurea and/or polyurethane. The second material is preferably present in an amount less than 3 wt. %, preferably less than 1 wt. % based on the total weight of the microcapsule slurry.

As non-limiting examples, the shell of microcapsules can be aminoplast-based, polyurea-based or polyurethane-based. The shell of the microcapsules can also be hybrid, namely organic-inorganic such as a hybrid shell composed of at least two types of inorganic particles that are cross-linked, or yet a shell resulting from the hydrolysis and condensation reaction of a polyalkoxysilane macro-monomeric composition.

According to an aspect, the shell of the microcapsules comprises an aminoplast copolymer, such as melamine-formaldehyde or urea-formaldehyde or cross-linked melamine formaldehyde or melamine glyoxal.

According to another aspect the shell of the microcapsules is polyurea-based made from, for example but not limited to isocyanate-based monomers and amine-containing crosslinkers such as guanidine carbonate and/or guanazole. Certain polyurea microcapsules comprise a polyurea wall which is the reaction product of the polymerisation between at least one polyisocyanate comprising at least two isocyanate functional groups and at least one reactant selected from the group consisting of an amine (for example a water-soluble guanidine salt and guanidine); a colloidal stabilizer or emulsifier; and an encapsulated perfume. However, the use of an amine can be omitted. According to a particular aspect, the colloidal stabilizer includes an aqueous solution of between 0.1% and 0.4% of polyvinyl alcohol, between 0.6% and 1% of a cationic copolymer of vinylpyrrolidone and of a quaternized vinylimidazol (all percentages being defined by weight relative to the total weight of the colloidal stabilizer). According to another aspect, the emulsifier is an anionic or amphiphilic biopolymer, which may be, in one aspect, chosen from the group consisting of gum Arabic, soy protein, gelatin, sodium caseinate and mixtures thereof.

According to another embodiment, the microcapsule wall material of the microcapsules may comprise any suitable resin and especially including melamine, glyoxal, polyurea, polyurethane, polyamide, polyester, etc. Suitable resins include the reaction product of an aldehyde and an amine, suitable aldehydes include, formaldehyde and glyoxal. Suitable amines include melamine, urea, benzoguanamine, glycoluril, and mixtures thereof. Suitable melamines include, methylol melamine, methylated methylol melamine, imino melamine and mixtures thereof. Suitable ureas include, dimethylol urea, methylated dimethylol urea, urea-resorcinol, and mixtures thereof. Suitable materials for making may be obtained from one or more of the following companies Solutia Inc. (St Louis, Missouri U.S.A.), Cytec Industries (West Paterson, New Jersey U.S.A.), Sigma-Aldrich (St. Louis, Missouri U.S.A.).

According to another embodiment, the microcapsules is a one-shell aminoplast core-shell microcapsule obtainable by a process comprising the steps of:
1) admixing a perfume oil with at least a polyisocyanate having at least two isocyanate functional groups to form an oil phase;
2) dispersing or dissolving into water an aminoplast resin and optionally a stabilizer to form a water phase;
3) preparing an oil-in-water dispersion, wherein the mean droplet size is comprised between 1 and 100 microns, by admixing the oil phase and the water phase;
4) performing a curing step to form the wall of said microcapsule; and
5) optionally drying the final dispersion to obtain the dried core-shell microcapsule.

According to an embodiment, the microcapsules is a formaldehyde-free capsule. A typical process for the preparation of aminoplast formaldehyde-free microcapsules slurry comprises the steps of
1) preparing an oligomeric composition comprising the reaction product of, or obtainable by reacting together:
   a. a polyamine component in the form of melamine or of a mixture of melamine and at least one C₁-C₄ compound comprising two NH2 functional groups;
   b. an aldehyde component in the form of a mixture of glyoxal, a C₄₋₆ 2,2-dialkoxy-ethanal and optionally a glyoxalate, said mixture having a molar ratio glyoxal/C₄₋₆ 2,2-dialkoxy-ethanal comprised between 1/1 and 10/1; and
   c. a protic acid catalyst;
2) preparing an oil-in-water dispersion, wherein the droplet size is comprised between 1 and 600 microns, and comprising:
   a. an oil;
   b. a water medium;
   c. at least an oligomeric composition as obtained in step 1;
   d. at least a cross-linker selected amongst:
      i. C₄-C₁₂ aromatic or aliphatic di- or tri-isocyanates and their biurets, triurets, trimmers, trimethylol propane-adduct and mixtures thereof; and/or
      ii. a di- or tri-oxiran compounds of formula:
         A-(oxiran-2-ylmethyl)ₙ
         wherein ₙ stands for 2 or 3 and 1 represents a C₂-C₆ group optionally comprising from 2 to 6 nitrogen and/or oxygen atoms;
   e. optionally a C₁-C₄ compounds comprising two NH₂ functional groups;
3) Heating the dispersion; and
4) Cooling the dispersion.

In another particular embodiment, the microcapsule comprises
an oil-based core comprising a hydrophobic material, preferably perfume,
optionally an inner shell made of a polymerized polyfunctional monomer;
a biopolymer shell comprising a protein, wherein at least one protein is cross-linked.

According to a particular embodiment, the protein is chosen in the group consisting of milk proteins, caseinate salts such as sodium caseinate or calcium caseinate, casein, whey protein, hydrolyzed proteins, gelatins, gluten, pea protein, soy protein, silk protein and mixtures thereof, preferably sodium caseinate, most preferably sodium caseinate

According to a particular embodiment, the protein comprises sodium caseinate and a globular protein, preferably chosen in the group consisting of whey protein, beta-lactoglobulin, ovalbumine, bovine serum albumin, vegetable proteins, and mixtures thereof.

The protein is preferably a mixture of sodium caseinate and whey protein.

According to a particular embodiment, the biopolymer shell comprises a crosslinked protein chosen in the group consisting of sodium caseinate and/or whey protein.

According to a particular embodiment, the microcapsules slurry comprises at least one microcapsule made of:
an oil-based core comprising the hydrophobic material, preferably perfume;
an inner shell made of a polymerized polyfunctional monomer; preferably a polyisocyanate having at least two isocyanate functional groups a biopolymer shell comprising a protein, wherein at least one protein is cross-linked; wherein the protein contains preferably a mixture comprising sodium caseinate and a globular protein, preferably whey protein.
optionally at least an outer mineral layer.

According to an embodiment, sodium caseinate and/or whey protein is (are) cross-linked protein(s).

The weight ratio between sodium caseinate and whey protein is preferably comprised between 0.01 and 100, preferably between 0.1 and 10, more preferably between 0.2 and 5.

In another particular embodiment, the microcapsules is a polyamide core-shell polyamide microcapsule comprising:
an oil-based core comprising comprising a hydrophobic material, preferably perfume, and
a polyamide shell comprising or being obtainable from:
   an acyl chloride,
   a first amino compound, and
   a second amino compound.

According to a particular embodiment, the microcapsules comprises:
an oil-based core comprising a hydrophobic material, preferably perfume, and a polyamide shell comprising or being obtainable from:
   an acyl chloride, preferably in an amount comprised between 5 and 98%, preferably between 20 and 98%, more preferably between 30 and 85% w/w
   a first amino compound, preferably in an amount comprised between 1% and 50% w/w, preferably between 7 and 40% w/w;
   a second amino compound, preferably in an amount comprised between 1% and 50% w/w, preferably between 2 and 25% w/w
   a stabilizer, preferably a biopolymer, preferably in an amount comprised between 0 and 90%, preferably between 0.1 and 75%, more preferably between 1 and 70%.

According to a particular embodiment, the microcapsules comprises:
an oil-based core comprising a hydrophobic material, preferably perfume, and
a polyamide shell comprising or being obtainable from:
   an acyl chloride,
   a first amino-compound being an amino-acid, preferably chosen in the group consisting of L-Lysine, L-Arginine, L-Histidine, L-Tryptophane and/or mixture thereof.
   a second amino.compound chosen in the group consisting of ethylene diamine, diethylene triamine, cystamine and/or mixture thereof, and
   a biopolymer chosen in the group consisting of casein, sodium caseinate, bovin serum albumin, whey protein, and/or mixture thereof.

The acyl chloride as defined above can have the following formula (I)

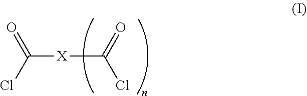

wherein n is an integer varying between 1 and 8, preferably between 1 and 6, more preferably between 1 and 4, and
wherein X is an (n+1)-valent C₂ to C₄₅ hydrocarbon group optionally comprising at least one group selected from (i) to (xi)

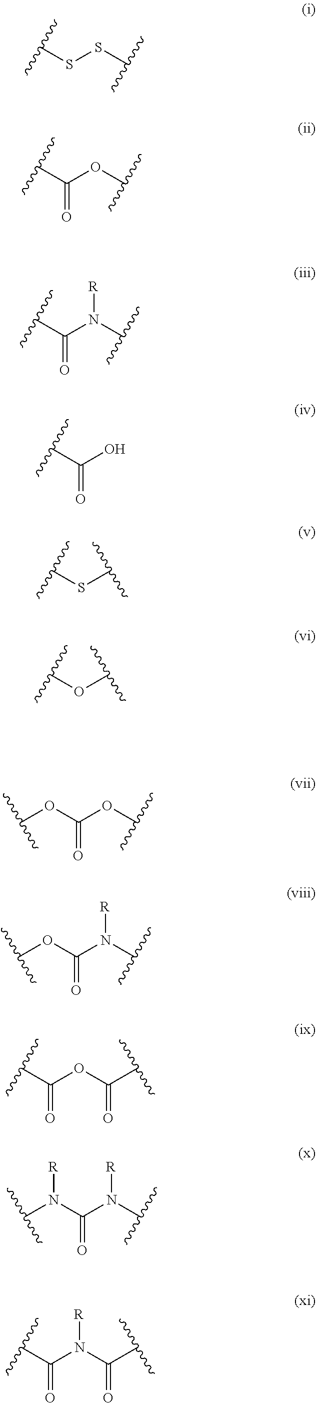

wherein R is a hydrogen atom or an alkyl group such as a methyl or an ethyl group, preferably a hydrogen atom.

It is understood that by “ . . . hydrocarbon group . . . ” it is meant that said group consists of hydrogen and carbon atoms and can be in the form of an aliphatic hydrocarbon, i.e. linear or branched saturated hydrocarbon (e.g. alkyl group), a linear or branched unsaturated hydrocarbon (e.g. alkenyl or alkynil group), a saturated cyclic hydrocarbon (e.g. cycloalkyl) or an unsaturated cyclic hydrocarbon (e.g. cycloalkenyl or cycloalkynyl), or can be in the form of an aromatic hydrocarbon, i.e. aryl group, or can also be in the form of a mixture of said type of groups, e.g. a specific group may comprise a linear alkyl, a branched alkenyl (e.g. having one or more carbon-carbon double bonds), a (poly)cycloalkyl and an aryl moiety, unless a specific limitation to only one type is mentioned. Similarly, in all the embodiments of the invention, when a group is mentioned as being in the form of more than one type of topology (e.g. linear, cyclic or branched) and/or being saturated or unsaturated (e.g. alkyl, aromatic or alkenyl), it is also meant a group which may comprise moieties having any one of said topologies or being saturated or unsaturated, as explained above. Similarly, in all the embodiments of the invention, when a group is mentioned as being in the form of one type of saturation or unsaturation, (e.g. alkyl), it is meant that said group can be in any type of topology (e.g. linear, cyclic or branched) or having several moieties with various topologies.

It is understood that with the term “ . . . a hydrocarbon group, optionally comprising . . . ” it is meant that said hydrocarbon group optionally comprises heteroatoms to form ether, thioether, amine, nitrile or carboxylic acid groups and derivatives (including for example esters, acids, amide). These groups can either substitute a hydrogen atom of the hydrocarbon group and thus be laterally attached to said hydrocarbon, or substitute a carbon atom (if chemically possible) of the hydrocarbon group and thus be inserted into the hydrocarbon chain or ring.

According to a particular embodiment, the acyl chloride is chosen from the group consisting of benzene-1,3,5-tricarbonyl trichloride (trimesoyl trichloride), benzene-1,2,4-tricarbonyl trichloride, benzene-1,2,4,5-tetracarbonyl tetrachloride, cyclohexane-1,3,5-tricarbonyl trichloride, isophthalyol dichloride, diglycolyl dichloride, terephthaloyl chloride, fumaryl dichloride, adipoyl chloride, succinic dichloride, propane-1,2,3-tricarbonyl trichloride, cyclohexane-1,2,4,5-tetracarbonyl tetrachloride, 2,2′-disulfanediyldisuccinyl dichloride, 2-(2-chloro-2-oxo-ethyl)sulfanylbutanedioyl dichloride, (4-chloro-4-oxobutanoyl)-L-glutamoyl dichloride, (S)-4-((1,5-dichloro-1,5-dioxopentan-2-yl)amino)-4-oxobutanoic acid, 2,2-bis[(4-chloro-4-oxo-butanoyl)oxymethyl]butyl 4-chloro-4-oxo-butanoate, [2-[2,2-bis[(4-chloro-4-oxo-butanoyl)oxymethyl]butoxymethyl]-2-[(4-chloro-4-oxo-butanoyl)oxymethyl]butyl]4-chloro-4-oxo-butanoate, 2,2-bis[(2-chlorocarbonylbenzoyl)oxymethyl]butyl 2-chlorocarbonyl-benzoate, [2-[2,2-bis[(2-chlorocarbonylbenzoyl)oxymethyl]butoxymethyl]-2-[(2-chlorocarbonylbenzoyl)oxymethyl]butyl]2-chlorocarbonylbenzoate, 4-(2,4,5-trichlorocarbonylbenzoyl)oxybutyl 2,4,5-trichlorocarbonyl-benzoate, propane-1,2,3-triyl tris(4-chloro-4-oxobutanoate), propane-1,2-diyl bis(4-chloro-4-oxobutanoate) and mixtures thereof.

According to another aspect, the shell of the microcapsules is polyurea- or polyurethane-based. Examples of processes for the preparation of polyurea and polyurethane-based microcapsule slurry are for instance described in International Patent Application Publication No. WO2007/004166, European Patent Application Publication No. EP 2300146, and European Patent Application Publication No. EP25799. Typically, a process for the preparation of polyurea or polyurethane-based microcapsule slurry include the following steps:
a) Dissolving at least one polyisocyanate having at least two isocyanate groups in an oil to form an oil phase;
b) Preparing an aqueous solution of an emulsifier or colloidal stabilizer to form a water phase;
c) Adding the oil phase to the water phase to form an oil-in-water dispersion, wherein the mean droplet size is comprised between 1 and 500 μm, preferably between 5 and 50 μm; and
d) Applying conditions sufficient to induce interfacial polymerisation and form microcapsules in form of a slurry.

In a particular embodiment, the shell material is a biodegradable material.

In a particular embodiment, the shell has a biodegradability of at least 60%, preferably at least 65%, 70%, 75%, 80%, 85%, 90%, 95% or 98%, within 60 days according to OECD301F.

In a particular embodiment, the core-shell microcapsule has a biodegradability of at least 40%, preferably at least 60%, preferably at least 65%, 70%, 75%, 80%, 85%, 90%, 95% or 98% within 60 days according to OECD301F.

Thereby it is understood that the core-shell microcapsule including all components, such as the core, shell and optionally coating may have a biodegradability of at least 40%, preferably at least 60%, preferably at least 65%, 70%, 75%, 80%, 85%, 90%, 95% or 98% within 60 days according to OECD301F.

In a particular embodiment, the oil core, preferably perfume oil, has a biodegradability of at least 40%, preferably at least 60%, preferably at least 65%, 70%, 75%, 80%, 85%, 90%, 95% or 98% within 60 days according to OECD301F.

OECD301F is a standard test method on the biodegradability from the Organization of Economic Co-operation and Development.

A typical method for extracting the shell for measuring the biodegradability is disclosed in Gasparini and all in Molecules 2020, 25,718.

### Mineral Layer

According to the invention, the microcapsule comprises a mineral layer on the terminating charged functional surface. According to an embodiment, the terminating functional surface is anionic and can be obtained by using an anionic emulsifier with optionally a polyelectrolyte scaffolding as defined above or by using a cationic emulsifier with at least one anionic polyelectrolyte layer.

According to the invention, the mineral layer comprises at least one salt chosen from the group consisting of barium salt, strontium salt, magnesium salt, and mixtures thereof.

According to an embodiment, the mineral layer comprises a salt chosen from the group consisting of barium sulfate, strontium sulfate, strontium carbonate, strontium phosphate, and mixtures thereof.

According to an embodiment, the mineral layer does not comprise a material chosen in the group consisting of iron oxides, iron oxyhydroxide, titanium oxides, zinc oxides, calcium carbonates, calcium phosphates and mixtures thereof.

According to an embodiment, the mineral layer does not comprise silicon oxides.

Another object of the invention is a core-shell microcapsule powder obtained by drying the core-shell microcapsule slurry as defined above.

Another object of the invention is a solid particle comprising:
a carrier material, preferably a polymeric carrier material chosen in the group consisting of polyvinyl acetate, polyvinyl alcohol, dextrins, natural or modified starch, vegetable gums, pectins, xanthans, alginates, carragenans, cellulose derivatives and mixtures thereof, and
microcapsules as defined above entrapped in said carrier material, and
optionally free perfume entrapped in said carrier material.

Solid particle as defined above and microcapsule powder can be used indifferently in the present invention.

### Optional Components

When microcapsules are in the form of a slurry, the microcapsule slurry can comprise auxiliary ingredients selected from the group of thickening agents/rheology modifiers, antimicrobial agents, opacity-building agents, mica particles, salt, pH stabilizers/buffering ingredients, preferably in an amount comprised between 0 and 15% by weight based on the total weight of the slurry.

According to another embodiment, the microcapsule slurry of the invention comprises additional free (i.e non-encapsulated) perfume, preferably in an amount comprised between 5 and 50% by weight based on the total weight of the slurry.

### Process for the Preparation of the Mineralized Core-Shell Microcapsule Slurry

Another object of the present invention is a process a process for preparing a mineralized core-shell microcapsule slurry as defined above comprising the steps of:
(i) Preparing a core-shell microcapsule slurry comprising microcapsules having a terminating charged functional surface;
(ii) Adsorption of at least one mineral precursor on the charged surface;
(iii) Applying conditions suitable to induce crystal growth of the mineral on the charged surface to form a mineral layer,
wherein the mineral precursor is adsorbed on the charged surface by incubating the core-shell microcapsule slurry obtained in step (i) in at least one mineral precursor solution, wherein the mineral precursor solution is chosen in the group consisting of barium salt solution, strontium salt solution, magnesium salt solution, phosphate-based salt solution, sulfate-based salt solution, carbonate-based salt solution and mixtures thereof.

According to a particular embodiment, the mineral precursor is chosen in the group consisting of Barium Nitrate, Barium Chloride, Barium Bromide, Barium Iodide, Barium Chlorate, Barium Hydroxide, Strontium Nitrate, Strontium Chloride, Strontium Iodide, Strontium Chlorate, Sodium Sulfate, Potassium Sulfate, Sodium Carbonate, Potassium Carbonate, Ammonium Carbonate, Sodium Phosphate, Potassium Phosphate, Ammonium phosphate and mixtures thereof.

Sodium phosphate used in the present invention can be monobasic (NaH₂PO₄), dibasic (Na₂HPO₄) or tribasic (Na₃PO₄).

Potassium phosphate used in the present invention can be monobasic (KH₂PO₄), dibasic (K₂HPO₄) or tribasic (K₃PO₄).

Ammonium phosphate used in the present invention can be monobasic ((NH₄)H₂PO₄), dibasic ((NH₄)₂HPO₄), or tribasic ((NH₄)₃PO₄).

According to an embodiment, the mineral precursor is chosen in the group consisting of Barium Nitrate, Barium Chloride, Barium Bromide, Barium Iodide, Barium Chlorate, Barium Hydroxide, Strontium Nitrate, Strontium Chloride, Strontium Iodide, Strontium Chlorate, Sodium Sulfate, Potassium Sulfate, Sodium Carbonate, Potassium Carbonate, ammonium carbonate, sodium phosphate (monobasic) (NaH₂PO₄), sodium phosphate (dibasic) (Na₂HPO₄), sodium phosphate (tribasic): Na₃PO₄, Potassium phosphate (monobasic): KH₂PO₄, Potassium phosphate (dibasic) (K₂HPO₄), potassium phosphate (tribasic) (K₃PO₄), ammonium phosphate (monobasic) ((NH₄)H₂PO₄), ammonium phosphate (dibasic) ((NH₄)₂HPO₄), ammonium phosphate (tribasic) ((NH₄)₃PO₄) and mixtures thereof.

According to an embodiment, in step ii) consists of the adsorption of two mineral precursors on the charged surface.

The water-soluble carbonate-based salt can be chosen in the group consisting of sodium, potassium and ammonium-based carbonates.

### Step (i) Preparing a Core-Shell Microcapsule Slurry Comprising Microcapsules Having a Terminating Charged Functional Surface

According to an embodiment, the polymeric shell is formed by interfacial polymerisation in the presence of a charged emulsifier.

One of the essential features of the present invention is that the polymeric shell has a terminating charged functional surface on which a mineral precursor will be adsorbed in step (ii). Different ways can be used to impart such charged surface on the polymeric shell.

According to a particular embodiment, the terminating charged functional surface is a terminating anionic functional surface.

### Emulsifier=Anionic Emulsifier

According to a first embodiment, the charged emulsifier is an anionic emulsifier and forms an anionic surface once the interfacial polymerization is completed.

The anionic emulsifier can be amphiphilic materials, colloidal stabilizers or biopolymers.

According to an embodiment, the anionic emulsifier is selected from the group consisting of polyvinyl alcohol, polyvinyl pyrrolidone, gum acacia, casein, sodium caseinate, soy protein, rice protein, whey protein, white egg albumin, gelatin, bovine serum albumin, hydrolyzed soy protein, hydrolyzed sericin, pseudocollagen, silk protein, sericin powder, gelatin and mixtures thereof.

According to an embodiment, gum acacia is preferred.

According to an embodiment, sodium caseinate and/or whey protein is preferred.

According to a particular embodiment, the anionic surface (formed by the anionic emulsifier) is the terminating anionic functional surface on which a mineral precursor will be adsorbed in step (ii).

However, to improve the bonding of mineral precursor on the anionic surface, step (i) can further comprise an additional step consisting of adding a polyelectrolyte scaffolding composed of oppositely-charge polyelectrolyte layer once the microcapsules are formed.

Thus, according to a particular embodiment, the polyelectrolyte scaffolding including at least one cationic polyelectrolyte layer and at least one anionic polyelectrolyte layer, the terminating layer being an anionic polyelectrolyte layer to form the terminating anionic functional surface of the shell.

According to this embodiment, the first layer of the polyelectrolyte scaffolding is a cationic polyelectrolyte layer disposed on the anionic surface (formed by the anionic emulsifier) and the last layer of the polyelectrolyte scaffolding is an anionic polyelectrolyte layer to form the terminating anionic functional surface on which on which a mineral precursor will be adsorbed in step (ii).

The number of layers of the polyelectrolyte scaffolding is not particularly limited.

According to a particular embodiment, the polyelectrolyte scaffolding consists of two pairs of oppositely charged polyelectrolytes layers.

It means that according to this embodiment, at the end of step (i), the microcapsule according to the invention comprises the following successive layers on the polymeric shell, a first cationic polyelectrolyte layer on the anionic surface (formed by the anionic emulsifier), a first negative polyelectrolyte layer, a second cationic polyelectrolyte layer, a second negative polyelectrolyte layer (forming the terminating anionic functional surface).

### Emulsifier=Cationic Emulsifier

According to a second embodiment, the charged emulsifier is a cationic emulsifier that forms a cationic surface when the interfacial polymerization is completed, and wherein step (i) further comprises a step of coating at least one anionic polyelectrolyte layer on the cationic surface to form core-shell microcapsule having a terminating anionic functional surface.

According to an embodiment, the cationic emulsifier is obtained by mixing a weakly anionic emulsifier (such as PVOH) with a strongly charged cationic polymer or polyquaternium (such as Salcare® SC-60 by BASF).

As non-limiting examples of cationic emulsifiers, one may cite for example cationic functionalized polyvinyl alcohol (as an example, cationic C-506 by Kuraray) or chitosan at an appropriate pH (typically at a weakly acidic pH (approximately pH 6.5).

According to a particular embodiment, the anionic surface (formed by the anionic polyelectrolyte layer) is the terminating anionic functional surface on which a mineral precursor will be adsorbed in step (ii).

According to another embodiment, at least one cationic polyelectrolyte layer and at least a second anionic polyelectrolyte layer are deposited successively on the anionic polyelectrolyte layer.

However, this embodiment is not limited to only one pair of opposite polyelectrolyte layers but includes 2, 3, 4 or even more of pair of opposite polyelectrolyte layers, with the proviso that the last polyelectrolyte layer is an anionic polyelectrolyte layer to form the terminating anionic functional surface.

According to an embodiment, the cationic polyelectrolyte layer is chosen in the group consisting of poly(allylamine hydrochloride), poly-L-lysine and chitosan.

According to another embodiment, the anionic polyelectrolyte layer is chosen in the group consisting of poly(sodium 4 styrene sulfonate) (PSS), polyacrylic acid, polyethylene imine, humic acid, carrageenan, gum acacia, and mixtures thereof.

According to a particular embodiment, the anionic polyelectrolyte layer is PSS.

The preparation of an aqueous slurry of core-shell microcapsules is well known from a skilled person in the art and has been disclosed above.

According to an embodiment, prior the step (ii), microcapsules are rinsed to remove the excess of emulsifier. Microcapsules can be rinsed for example by centrifugation and resuspended in water after withdrawing the supernatant.

Step (ii) and Step (iii)—Mineralization and Crystal Growth

Without being bound by theory, it is believed that the charged terminating surface is providing functional anchoring sites and a high local density of charge groups and nucleation sites onto the surface of the microcapsules resulting in improved adsorption of mineral precursor species followed by initiation of the crystal growth process through in-situ addition of a precipitating species.

Mineral precursors are adsorbed to the surface of microcapsules by incubating the charged capsules in at least one solution containing oppositely charged mineral precursor, providing sufficient agitation and time to allow for complete coverage of capsule surfaces. Removal of excess precursor from solution to prevent generation of free crystalline material in solution can be done and is followed by initiation of the crystal growth process through in-situ addition of a precipitating species.

The person skilled in the art will be able to select suitable conditions for the crystal growth process (for example, precursor selection, reaction conditions, the solution concentrations, incubation times, agitation speeds, temperatures and pH conditions). Typically:
mineralization occurs at room temperature,
incubation of precursor takes place from 24-72 hours,
the nature of the precipitation species depends on the nature of the precursor.

According to the invention, the mineral precursor solution is chosen in the group consisting of a barium salt solution (comprising barium ions as precursor), strontium salt solution (comprising strontium ions as precursor), magnesium salt solution (comprising magnesium ions as precursor), phosphate-based salt solution (comprising phosphate ions as precursor), sulfate-based salt solution (comprising sulfate ions as precursor), carbonate-based salt solution (comprising carbonate ions as precursor) and mixtures thereof.

The water-soluble barium-based salt can be chosen in the group consisting of Barium Nitrate, Barium Chloride, Barium Bromide, Barium Iodide, Barium Chlorate, Barium Hydroxide and mixtures thereof

The water-soluble strontium-based salt can be chosen in the group consisting of Strontium Nitrate, Strontium Chloride, Strontium Iodide, Strontium Chlorate and mixtures thereof

The water-soluble magnesium-based salt can be chosen in the group consisting of Magnesium Nitrate, Magnesium Chloride, Magnesium Sulfate, Magnesium Iodide, Magnesium Bromide, Magnesium Chlorate and mixtures thereof.

The water-soluble phosphate-based salt can be chosen in the group consisting of sodium phosphate (monobasic) (NaH₂PO₄), sodium phosphate (dibasic) (Na₂HPO₄), sodium phosphate (tribasic): Na₃PO₄, Potassium phosphate (monobasic): KH₂PO₄, Potassium phosphate (dibasic) (K₂HPO₄), potassium phosphate (tribasic) (K₃PO₄), ammonium phosphate (monobasic) ((NH₄)H₂PO₄), ammonium phosphate (dibasic) ((NH₄)₂HPO₄), ammonium phosphate (tribasic) ((NH₄)₃PO₄) and mixtures thereof.

The water-soluble carbonate-based salt can be chosen in the group consisting of sodium, potassium and ammonium-based carbonates.

According to an embodiment, the mineral precursor does not comprise silicon oxides.

According to an embodiment, the mineral precursor solution is not an iron (II) sulfate solution, an iron (III) chloride solution, a calcium-based salt solution, a titanium-based precursor solution, a zinc-based precursor solution, and mixtures thereof.

It should be understood that the charge of the mineral precursor used in step (ii) of the process is driven by the charge of the terminating surface of the microcapsules.

According to another embodiment, microcapsules are introduced sequentially in at least two solutions comprising respectively at least one precursor. Preferably, the first solution comprises water-soluble barium-based salt including a barium precursor and the second solution comprises water-soluble sulfate-based salt including a sulfate precursor. Addition order could change according to the selection and charge of the underlying terminating layer.

According to another embodiment, microcapsules are introduced sequentially in at least two solutions comprising respectively at least one precursor. Preferably, the first solution comprises water-soluble strontium-based salt including a strontium precursor and the second solution comprises water-soluble phosphate-based salt including a phosphate precursor. Addition order could change according to the selection and charge of the underlying terminating layer.

According to a particular embodiment, the process for the preparation of the microcapsule slurry comprises the following steps:
a) dissolving at least one a polyisocyanate having at least two isocyanate groups in an oil comprising a hydrophobic material to form an oil phase;
b) preparing an aqueous solution of a charged emulsifier to form a water phase, wherein the charged emulsifier is an anionic emulsifier or a cationic emulsifier;
c) adding the oil phase to the water phase to form an oil-in-water dispersion;
d) applying conditions suitable to induce interfacial polymerization to form core/shell microcapsules in the form of a slurry, wherein:
   the shell has an anionic surface when the emulsifier used in step b) is an anionic emulsifier; or
   the shell has a cationic surface when the emulsifier used in step b) is a cationic emulsifier;
e) coating at least one anionic polyelectrolyte layer on the cationic surface when the emulsifier is a cationic emulsifier to form an anionic surface;
f) Optionally, dilution or removal of excess emulsifier;
g) adsorption of a mineral precursor on the anionic surface as defined above;
h) applying conditions suitable to induce crystal growth of the mineral on the anionic surface; and
i) optionally drying the slurry.

According to this embodiment, the process comprises the preparation of an oil phase by dissolving a polyisocyanate having at least two isocyanate groups in an oil comprising a hydrophobic material as defined above.

According to a preferred embodiment of the invention, there is used an amount of between 10 and 60%, more preferably between 20 and 50% of oil in the process of the invention, these percentages being defined by weight relative to the total weight of the obtained microcapsule slurry.

Suitable polyisocyanates used according to the invention include aromatic polyisocyanate, aliphatic polyisocyanate and mixtures thereof. Said polyisocyanate comprises at least 2, preferably at least 3 but may comprise up to 6, or even only 4, isocyanate functional groups. According to a particular embodiment, a triisocyanate (3 isocyanate functional group) is used.

According to one embodiment, said polyisocyanate is an aromatic polyisocyanate.

The term “aromatic polyisocyanate” is meant here as encompassing any polyisocyanate comprising an aromatic moiety. Preferably, it comprises a phenyl, a toluyl, a xylyl, a naphthyl or a diphenyl moiety, more preferably a toluyl or a xylyl moiety. Preferred aromatic polyisocyanates are biurets, polyisocyanurates and trimethylol propane adducts of diisocyanates, more preferably comprising one of the above-cited specific aromatic moieties. More preferably, the aromatic polyisocyanate is a polyisocyanurate of toluene diisocyanate (commercially available from Bayer under the tradename Desmodur® RC), a trimethylol propane-adduct of toluene diisocyanate (commercially available from Bayer under the tradename Desmodur® L75), a trimethylol propane-adduct of xylylene diisocyanate (commercially available from Mitsui Chemicals under the tradename Takenate® D-110N). In a most preferred embodiment, the aromatic polyisocyanate is a trimethylol propane-adduct of xylylene diisocyanate.

According to another embodiment, said polyisocyanate is an aliphatic polyisocyanate. The term “aliphatic polyisocyanate” is defined as a polyisocyanate which does not comprise any aromatic moiety. Preferred aliphatic polyisocyanates are a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100), among which a biuret of hexamethylene diisocyanate is even more preferred.

According to another embodiment, the at least one polyisocyanate is in the form of a mixture of at least one aliphatic polyisocyanate and of at least one aromatic polyisocyanate, both comprising at least two or three isocyanate functional groups, such as a mixture of a biuret of hexamethylene diisocyanate with a trimethylol propane-adduct of xylylene diisocyanate, a mixture of a biuret of hexamethylene diisocyanate with a polyisocyanurate of toluene diisocyanate and a mixture of a biuret of hexamethylene diisocyanate with a trimethylol propane-adduct of toluene diisocyanate. Most preferably, it is a mixture of a biuret of hexamethylene diisocyanate with a trimethylol propane-adduct of xylylene diisocyanate. Preferably, when used as a mixture the molar ratio between the aliphatic polyisocyanate and the aromatic polyisocyanate is ranging from 80:20 to 10:90.

The at least one polyisocyanate used in the process according to the invention is present in amounts representing from 1 to 15%, preferably from 2 to 8% and more preferably from 2 to 6% by weight of the oil phase.

The at least one polyisocyanate is dissolved in an oil, which in a particular embodiment contains a perfume or flavour. The oil can contain a further oil-soluble benefit agent to be co-encapsulated with the perfume and flavour with the purpose of delivering additional benefit on top of perfuming or taste-related. As non-limiting examples, ingredients such as cosmetic, skin caring, malodor counteracting, bactericide, fungicide, pharmaceutical or agrochemical ingredient, a diagnostic agent and/or an insect repellent or attractant and mixtures thereof can be used.

According to an embodiment, the process of the present invention includes the use of an anionic or amphiphilic biopolymer in the preparation of the aqueous phase. Those materials defined above include in particular proteins and polysaccharides. The biopolymer is preferably comprised in an amount ranging from 0.1 to 5.0% by weight of the microcapsule slurry, preferably between 0.5 and 2 wt % of the microcapsule slurry.

The above ranges also apply when the process includes the use of a charged emulsifier.

According to a first embodiment, the charged emulsifier used in step b) is an anionic emulsifier and forms an anionic surface when step d) is completed.

According to an embodiment, the anionic emulsifier is selected from the group consisting of polyvinyl alcohol, polyvinyl pyrilidone, gum acacia, casein, sodium caseinate, soy protein, rice protein, whey protein, white egg albumin, gelatin, bovine serum albumin, hydrolyzed soy protein, hydrolyzed sericin, pseudocollagen, silk protein, sericin powder, sugar beet pectin, gelatin and mixtures thereof.

According to a particular embodiment, the anionic emulsifier is gum acacia.

According to a second embodiment, a cationic emulsifier is used in step b) and forms a cationic surface when step d) is completed.

As non-limiting examples of cationic emulsifiers, one may cite for example cationically modified polyvinyl alcohol (as an example, cationic C-506 by Kuraray) or chitosan.

According to this embodiment, the process further comprises a step consisting in coating an anionic polyelectrolyte layer to impart a negatively charged surface necessary to induce the crystal growth of the mineral.

To enhance the adsorption of mineral precursors to the terminating anionic functional surface, said surface can be modified through the adsorption of a polyelectrolyte multilayered scaffolding.

Thus, according to an embodiment, the process comprises a further step after step d) or after step e), consisting in coating at least one cationic polyelectrolyte layer and at least one anionic polyelectrolyte layer, the terminating layer being an anionic polyelectrolyte layer to form the terminating anionic functional surface.

According to this embodiment, the cationic polyelectrolyte layer is disposed on the anionic surface and the anionic polyelectrolyte layer is the last layer to form the terminating anionic functional surface on which the mineral precursor is adsorbed.

Oppositely-charge polyelectrolytes may be sequentially coated onto microcapsules using layer-by-layer polyelectrolyte deposition in order to provide a multi-layered polyelectrolyte scaffold for adsorption of mineral precursors.

According to the invention, the number of layers of the polyelectrolyte scaffolding is not particularly limited.

According to a particular embodiment, the polyelectrolyte scaffolding consists of two pairs of oppositely charged polyelectrolytes layers.

It means that according to this embodiment, after step d) or step e), the process comprises:
applying a cationic polyelectrolyte layer C1 on the anionic layer;
applying an anionic polyelectrolyte layer A1 on the cationic polyelectrolyte layer C1,
applying a cationic polyelectrolyte layer C2 on the anionic polyelectrolyte layer A1;
applying an anionic polyelectrolyte layer A2 on the cationic polyelectrolyte layer C2, thereby forming the anionic terminating functional surface on which the mineral precursor is adsorbed.

According to an embodiment, the cationic polyelectrolyte layer is chosen in the group consisting of poly(allylamine hydrochloride), poly-L-lysine and chitosan.

According to another embodiment, the anionic polyelectrolyte layer is chosen in the group consisting of poly(sodium 4 styrene sulfonate) (PSS), polyacrylic acid, polyethylene imine, humic acid, carrageenan, gum acacia, and mixtures thereof.

According to a particular embodiment, the anionic polyelectrolyte layer is PSS.

According to a particular embodiment, the process comprises after step h) a further step consisting of hydrolysis of the mineral layer. This can be done for example by addition of sodium hydroxide.

Another object of the invention is a process for preparing a microcapsule powder comprising the steps as defined above and an additional step iii) consisting of submitting the slurry obtained in step iii) to a drying, like spray-drying, to provide the microcapsules as such, i.e. in a powdery form. It is understood that any standard method known by a person skilled in the art to perform such drying is also applicable. In particular the slurry may be spray-dried preferably in the presence of a polymeric carrier material such as polyvinyl acetate, polyvinyl alcohol, dextrins, natural or modified starch, vegetable gums, pectins, xanthans, alginates, carragenans or cellulose derivatives to provide microcapsules in a powder form.

According to a particular embodiment, the carrier material contains free perfume oil which can be the same or different from the perfume from the core of the microcapsules.

However, one may cite also other drying method such as the extrusion, plating, spray granulation, the fluidized bed, or even a drying at room temperature using materials (carrier, desiccant) that meet specific criteria as disclosed in WO2017/134179.

### Microcapsule Slurry/Microcapsule Powder

A microcapsule slurry or microcapsules obtainable by the process as defined above are also subjects of the present invention.

Another object of the invention is a microcapsule powder obtained by drying the microcapsule slurry defined above.

### Perfuming Composition and Consumer Products

The microcapsules of the invention can be used in combination with active ingredients. An object of the invention is therefore a composition comprising:
(i) microcapsules or microcapsule slurry as defined above;
(ii) an active ingredient, preferably chosen in the group consisting of a cosmetic ingredient, skin caring ingredient, perfume ingredient, flavor ingredient, malodour counteracting ingredient, bactericide ingredient, fungicide ingredient, pharmaceutical or agrochemical ingredient, a sanitizing ingredient, an insect repellent or attractant, and mixtures thereof.

### Perfumed Consumer Products

The capsules of the invention show a good performance in terms of stability in challenging medium.

Another object of the present invention is a perfuming composition comprising:
(i) microcapsules or microcapsule slurry as defined above, wherein the oil comprises a perfume;
(ii) at least one ingredient selected from the group consisting of a perfumery carrier, a perfumery co-ingredient and mixtures thereof;
(iii) optionally at least one perfumery adjuvant.

As liquid perfumery carriers one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the most commonly used. For the compositions which comprise both a perfumery carrier and a perfumery co-ingredient, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowano1® (origin: Dow Chemical Company). By “perfumery co-ingredient” it is meant here a compound, which is used in a perfuming preparation or a composition to impart a hedonic effect and which is not a microcapsule as defined above. In other words such a co-ingredient, to be considered as being a perfuming one, must be recognized by a person skilled in the art as being able to at least impart or modify in a positive or pleasant way the odor of a composition, and not just as having an odor.

The nature and type of the perfuming co-ingredients present in the perfuming composition do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulfurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release in a controlled manner various types of perfuming compounds. Co-ingredients may be chosen in the group consisting of 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, trans-3-(dodecylthio)-1-(2,6,6-trimethyl-3-cyclohexen-1-yl)-1-butanone, 2-(dodecylthio) octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl)succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy) undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl) naphthalene, (2-phenethoxyvinyl)benzene, 2-(1-((3,7-dimethyloct-6-en-1-yl)oxy) prop-1-en-2-yl) naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

By “perfumery adjuvant” we mean here an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

Preferably, the perfuming composition according to the invention comprises between 0.01 and 30% by weight of microcapsules as defined above.

The invention's microcapsules can advantageously be used in many application fields and used in consumer products. Microcapsules can be used in liquid form applicable to liquid consumer products as well as in powder form, applicable to powder consumer products.

According to a particular embodiment, the consumer product as defined above is liquid and comprises:
a) from 2 to 65% by weight, relative to the total weight of the consumer product, of at least one surfactant;
b) water or a water-miscible hydrophilic organic solvent; and
c) a microcapsule slurry or microcapsules as defined above,
d) optionally non-encapsulated perfume.

According to a particular embodiment, the consumer product as defined above is in a powder form and comprises:
a) from 2 to 65% by weight, relative to the total weight of the consumer product, of at least one surfactant;
b) a microcapsule powder as defined above.
c) optionally perfume powder that is different from the microcapsules defined above.

In the case of microcapsules including a perfume oil-based core, the products of the invention, can in particular be of used in perfumed consumer products such as product belonging to fine fragrance or “functional” perfumery. Functional perfumery includes in particular personal-care products including hair-care, body cleansing, skin care, hygiene-care as well as home-care products including laundry care, surface care and air care. Consequently, another object of the present invention consists of a perfumed consumer product comprising as a perfuming ingredient, the microcapsules defined above or a perfuming composition as defined above. The perfume element of said consumer product can be a combination of perfume microcapsules as defined above and free or non-encapsulated perfume, as well as other types of perfume microcapsules than those here-disclosed.

In particular a liquid consumer product comprising:
a) from 2 to 65% by weight, relative to the total weight of the consumer product, of at least one surfactant;
b) water or a water-miscible hydrophilic organic solvent; and
c) a perfuming composition as defined above is another object of the invention.

Also a powder consumer product comprising
(a) from 2 to 65% by weight, relative to the total weight of the consumer product, of at least one surfactant; and
(b) a perfuming composition as defined above is part of the invention.

The invention's microcapsules can therefore be added as such or as part of an invention's perfuming composition in a perfumed consumer product.

For the sake of clarity, it has to be mentioned that, by “perfumed consumer product” it is meant a consumer product which is expected to deliver among different benefits a perfuming effect to the surface to which it is applied (e.g. skin, hair, textile, paper, or home surface) or in the air (air-freshener, deodorizer etc.). In other words, a perfumed consumer product according to the invention is a manufactured product which comprises a functional formulation also referred to as “base”, together with benefit agents, among which an effective amount of microcapsules according to the invention.

The nature and type of the other constituents of the perfumed consumer product do not warrant a more detailed description here, which in any case would not be exhaustive, the skilled person being able to select them on the basis of his general knowledge and according to the nature and the desired effect of said product. Base formulations of consumer products in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Non-limiting examples of suitable perfumed consumer products can be a perfume, such as a fine perfume, a cologne, an after-shave lotion, a body-splash; a fabric care product, such as a liquid or solid detergent, tablets and unit dose (single or multi chambers), a fabric softener, a dryer sheet, a fabric refresher, an ironing water, or a bleach; a personal-care product, such as a hair-care product (e.g. a shampoo, hair conditioner, a coloring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product); an air care product, such as an air freshener or a “ready to use” powdered air freshener; or a home care product, such all-purpose cleaners, liquid or power or tablet dishwashing products, toilet cleaners or products for cleaning various surfaces, for example sprays & wipes intended for the treatment/refreshment of textiles or hard surfaces (floors, tiles, stone-floors etc.); a hygiene product such as sanitary napkins, diapers, toilet paper.

Another object of the invention is a consumer product comprising:
a personal care active base, and
microcapsules or a microcapsule slurry as defined above or the perfuming composition as defined above,
wherein the consumer product is in the form of a personal care composition.

Personal care active bases in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The personal care composition is preferably chosen in the group consisting of a hair-care product (e.g. a shampoo, hair conditioner, a coloring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product);

Another object of the invention is a consumer product comprising:
a home care or a fabric care active base, and
microcapsules or a microcapsule slurry as defined above or the perfuming composition as defined above,
wherein the consumer product is in the form of a home care or a fabric care composition.

Home care or fabric care active bases in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Preferably, the consumer product comprises from 0.1 to 15 wt %, more preferably between 0.2 and 5 wt % of the microcapsules of the present invention, these percentages being defined by weight relative to the total weight of the consumer product. Of course the above concentrations may be adapted according to the benefit effect desired in each product.

An object of the invention is a consumer product, preferably a home care or a fabric care consumer product comprising the microcapsules or the microcapsule slurry as defined above, wherein the consumer product has a pH less than 7.

An object of the invention is a consumer product, preferably a home care or a fabric care consumer product comprising the microcapsules or the microcapsule slurry as defined above, wherein the consumer product has a pH equals or greater than 7.

For liquid consumer product mentioned below, by “active base”, it should be understood that the active base includes active materials (typically including surfactants) and water.

For solid consumer product mention below, by “active base”, it should be understood that the active base includes active materials (typically including surfactants) and auxiliary agents (such as bleaching agents, buffering agent; builders; soil release or soil suspension polymers; granulated enzyme particles, corrosion inhibitors, antifoaming, sud suppressing agents; dyes, fillers, and mixtures thereof).

### Fabric Softener

An object of the invention is a consumer product in the form of a fabric softener composition comprising:
a fabric softener active base; preferably comprising at least one active material chosen in the group consisting of dialkyl quaternary ammonium salts, dialkyl ester quaternary ammonium salts (esterquats), Hamburg esterquat (HEQ), TEAQ (triethanolamine quat), silicones and mixtures thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Liquid Detergent

An object of the invention is a consumer product in the form of a liquid detergent composition comprising:
a liquid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Solid Detergent

An object of the invention is a consumer product in the form of a solid detergent composition comprising:
a solid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule powder or microcapsule slurry as defined above, preferably in an amount comprised between 0.05 to 15 wt %, more preferably between 0.1 and 5 wt % by weight based on the total weight of the composition,
optionally free perfume oil.

### Shampoo/Shower Gel

An object of the invention is a consumer product in the form of a shampoo or a shower gel composition comprising:
a shampoo or a shower gel active base; preferably comprising at least one active material chosen in the group consisting of sodium alkylether sulfate, ammonium alkylether sulfates, alkylamphoacetate, cocamidopropyl betaine, cocamide MEA, alkylglucosides and aminoacid based surfactants and mixtures thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Rinse-Off Conditioner

An object of the invention is a consumer product in the form of a rinse-off conditioner composition comprising:
a rinse-off conditioner active base; preferably comprising at least one active material chosen in the group consisting of cetyltrimonium chloride, stearyl trimonium chloride, benzalkonium chloride, behentrimonium chloride and mixture thereof, the active base being used preferably in an amount comprised between 85 and 99.95% by weight based on the total weight of the composition,
a microcapsule slurry or microcapsule as defined above, preferably in an amount comprised between 0.05 to 15 wt %, more preferably between 0.1 and 5 wt % by weight based on the total weight of the composition,
optionally free perfume oil.

### Solid Scent Booster

An object of the invention is a consumer product in the form of a solid scent booster composition comprising:
a solid carrier, preferably chosen in the group consisting of urea, sodium chloride, sodium sulphate, sodium acetate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, saccharides such as sucrose, mono-, di-, and polysaccharides and derivatives such as starch, cellulose, methyl cellulose, ethyl cellulose, propyl cellulose, polyols/sugar alcohols such as sorbitol, maltitol, xylitol, erythritol, and isomalt, PEG, PVP, citric acid or any water soluble solid acid, fatty alcohols or fatty acids and mixtures thereof,
a microcapsule slurry or microcapsules as defined above, in a powdered form, preferably in an amount comprised between 0.05 to 15 wt %, more preferably between 0.1 and 5 wt % by weight based on the total weight of the composition,
optionally free perfume oil.

### Liquid Scent Booster

An object of the invention is a consumer product in the form of a liquid scent booster composition comprising:
an aqueous phase,
a surfactant system essentially consisting of one or more than one non-ionic surfactant, wherein the surfactant system has a mean HLB between 10 and 14, preferably chosen in the group consisting of ethoxylated aliphatic alcohols, POE/PPG (polyoxyethylene and polyoxypropylene) ethers, mono and polyglyceryl esters, sucrose ester compounds, polyoxyethylene hydroxylesters, alkyl polyglucosides, amine oxides and combinations thereof;
a linker chosen in the group consisting of alcohols, salts and esters of carboxylic acids, salts and esters of hydroxyl carboxylic acids, fatty acids, fatty acid salts, glycerol fatty acids, surfactant having an HLB less than 10 and mixtures thereof, and
a microcapsule slurry or microcapsules as defined above, in the form of a slurry, preferably in an amount comprised between 0.05 to 15 wt %, more preferably between 0.1 and 5 wt % by weight based on the total weight of the composition,
optionally free perfume oil.

### Hair Coloration

An object of the invention is a consumer product in the form of an oxidative hair coloring composition comprising:
an oxidizing phase comprising an oxidizing agent and an alkaline phase comprising an alkakine agent, a dye precursor and a coupling compound; wherein said dye precursor and said coupling compound form an oxidative hair dye in the presence of the oxidizing agent, preferably in an amount comprised between 85 and 99.95% by weight based on the total weight of the composition,
microcapsules or microcapsule slurry as defined above, preferably in an amount comprised between 0.05 to 15 wt %, more preferably between 0.1 and 5 wt % by weight based on the total weight of the composition,
optionally free perfume oil

### Perfuming Composition

According to a particular embodiment, the consumer product is in the form of a perfuming composition comprising:
0.1 to 30%, preferably 0.1 to 20% of microcapsules or microcapsule slurry as defined previously,
0 to 40%, preferably 3-40% of perfume, and
20-90%, preferably 40-90% of ethanol, by weight based on the total weight of the perfuming composition.

### Flavored Consumer Products

End products are more particularly a food, pet-food or feed products.

As the particles of the invention comprise an hydrophobic coating they are particularly advantageous for dry food product susceptible to rehydrated like instant drinks (PSD, chocolate, coffee), confectionary like chewing gum, instant noodles or stock cubes. The particles of the invention are particularly advantageous to food product with a relatively high-water activity such as ready to use meal, meat analogs, microwave food, pasta boxes.

The particles of the invention can be used in vegetarian meat analogues or meat replacers, vegetarian burger, sausages, patties, chicken-imitate nuggets . . . , meat products (e.g. processed meat, poultry, beef, pork, ham, fresh sausage or raw meat preparations, spiced or marinated fresh meat or cured meat products, reformed meat) or extended meat products making use of a combination of animal and vegetable protein in varying ratios, often being coextruded or a mix between textured vegetable protein and animal protein.

Meat, for the purpose of the present invention, encompasses red meat, such as beef, pork, sheep, lamb, game and poultry, such as chicken, turkey, goose and duck. Preferably, the food of the present invention is meat selected from beef, poultry and pork.

Nevertheless, the particles of the invention can also be of particular interest in the following examples of products:
Baked goods (e.g. bread, dry biscuits, cakes, other baked goods),
Non-alcoholic beverages (e.g. carbonated soft drinks, bottled waters, sports/energy drinks, juice drinks, vegetable juices, vegetable juice preparations),
Alcoholic beverages (e.g. beer and malt beverages, spirituous beverages),
Instant beverages (e.g. instant vegetable drinks, powdered soft drinks, instant coffee and tea),
Cereal products (e.g. breakfast cereals, pre-cooked ready-made rice products, rice flour products, millet and sorghum products, raw or pre-cooked noodles and pasta products),
Milk products (e.g. fresh cheese, soft cheese, hard cheese, milk drinks, whey, butter, partially or wholly hydrolysed milk protein-containing products, fermented milk products, condensed milk and analogues),
Dairy based products (e.g. fruit or flavored yoghurt, ice cream, fruit ices)
Confectionary products (e.g. chewing gum, hard and soft candy)
Chocolate and compound coatings
Products based on fat and oil or emulsions thereof (e.g. mayonnaise, spreads, margarines, shortenings, remoulade, dressings, spice preparations),
Spiced, marinated or processed fish products (e.g. fish sausage, surimi),
Eggs or egg products (dried egg, egg white, egg yolk, custard),
Desserts (e.g. gelatins and puddings)
Products made of soya protein or other soya bean fractions (e.g. soya milk and products made therefrom, soya lecithin-containing preparations, fermented products such as tofu or tempeh or products manufactured therefrom, soya sauces),
Vegetable preparations (e.g. ketchup, sauces, processed and reconstituted vegetables, dried vegetables, deep frozen vegetables, pre-cooked vegetables, vegetables pickled in vinegar, vegetable concentrates or pastes, cooked vegetables, potato preparations),
Vegetarian meat replacer, vegetarian burger
Spices or spice preparations (e.g. mustard preparations, horseradish preparations), spice mixtures and, in particular seasonings which are used, for example, in the field of snacks.
Snack articles (e.g. baked or fried potato crisps or potato dough products, bread dough products, extrudates based on maize, rice or ground nuts),
Meat products (e.g. processed meat, poultry, beef, pork, ham, fresh sausage or raw meat preparations, spiced or marinated fresh meat or cured meat products, reformed meat),
Ready dishes (e.g. instant noodles, rice, pasta, pizza, tortillas, wraps) and soups and broths (e.g. stock, savory cube, dried soups, instant soups, pre-cooked soups, retorted soups), sauces (instant sauces, dried sauces, ready-made sauces, gravies, sweet sauces).

Preferably, the particles according to the invention shall be used in products selected from the group consisting of baked goods, instant beverages, cereal products, milk products, dairy-based products, products based on fat and oil or emulsions thereof, desserts, vegetable preparations, vegetarian meat replacer, spices and seasonings, snacks, meat products, ready dishes, soups and broths and sauces.

According to a particular embodiment, the flavored product is chosen group consisting of a meat- and/or fish-based food or analogue, a stock, a savory cube, a powder mix, a beef or pork based product, a seafood, surimi, instant noodles, rice, soups, sauces, ready-made meal, frozen or chilled pizza, pasta, potato flakes or fried, noodles, a potato/tortilla chip, a microwave popcorn, nuts, a pretzel, a rice cake, a rice cracker, fermented dairy analogue beverage, acidified dairy analogue beverage, non-fermented dairy analogue beverage, cheese or cheese analogue, yoghurt or yoghurt analogue, nutritional supplement, nutritional bar, cereal, ice cream, dairy-free ice cream, confectionary product, chewing gum, hard-boiled candy and powdered drinks.

According to one embodiment, the food, pet-food or feed product comprises between 0.01 and 10% by weight, preferably between 0.1 and 5% by weight of the particles of the invention.

Typically the food, pet-food or feed product further comprises proteins notably vegetable proteins or animal proteins, and mixtures thereof.

Advantageously the vegetable proteins are preferably selected among soy protein, corn, peas, canola, sunflowers, sorghum, rice, amaranth, potato, tapioca, arrowroot, chickpeas, lupins, canola, wheat, oats, rye, barley, and mixtures thereof.

The particles of the invention are particularly suitable for extruded and/or baked food, pet-food or feed products more particularly comprising animal and/or vegetable proteins. Typically, said extruded and/or baked food, pet-food or feed products may be selected among meat- and/or fish-based food or analogue and mixtures thereof (in other words, meat-based food and/or fish-based food or meat analogue or fish analogue and mixtures thereof); extruded and/or baked meat analogue or extruded and/or baked fish analogue are preferred. Non-limiting examples of extruded and/or baked food, pet-food or feed products are snack products or extruded vegetable proteins with the aim to texture the protein from which meat analogous (e.g. burgers) are prepared from. The powder composition can be added pre-extrusion or after extrusion to either, the non-extruded vegetable protein isolate/concentrate or to the textured vegetable protein from which a burger or nugget (etc.) is formed.

The invention will now be further described by way of examples. It will be appreciated that the invention as claimed is not intended to be limited in any way by these examples.

### EXAMPLES

### Example 1

Preparation of biopolymer-based capsules according to the invention

### Protocol 1

Microcapsules A1, B and C were prepared according to the following protocol.
1) Sodium caseinate and/or whey protein is dissolved in DI water at RT.
2) Calcium chloride (aqueous solution) is slowly added to the protein solution and stirred at RT for ˜15 min.
3) The emulsifier solution is combined with a perfume oil (see table 2) containing a polyisocyanate (Takenate® D-110N) and homogenized (10,000 rpm for 2 min).
4) The emulsion is then transferred to a reactor, pH adjusted to ˜6.5 w/NaOH, and heated to 45° C.
5) Transglutaminase (aqueous solution) is added to the reactor and it is stirred for 3 hr at 45° C.
6) The pH is adjusted to ˜5.4 w/HCl and then heated to 85° C.
7) The reactor is stirred at 85° C. for 60 min before cooling to RT.

TABLE 1
Microcapsule compositions

| Components | Wt % |
|---|---|
| Sodium Caseinate¹⁾ | 0.625 |
| Whey Protein²⁾ | 1.875 |
| CaCl₂•2H₂O | 0.5 |
| Perfume A³⁾ | 30 |
| Takenate D-110N⁴⁾ | 0.6 |
| Transglutaminase⁵⁾ | 1.0 |

¹⁾Ramsen Food and Dairy Products LLC
²⁾Agropur Dairy Cooperative
³⁾See table 2
⁴⁾Trimethylol propane-adduct of xylylene diisocyanate, origin: Mitsui Chemicals, Inc., Japan, 75% polyisocyanate/25% ethyl acetate
⁵⁾Activa TI ® origin: Ajinomoto

### Protocol 2

Microcapsules A2 were prepared according to the following protocol.

Benzene-1,3,5-tricarbonyle chloride (1.73 g) was dissolved in benzyl benzoate (5 g). Sodium caseinate (2 g) was dispersed in benzyl benzoate (5 g) and the dispersion was maintained under stirring at 60° C. for one hour. Both oil phases were mixed together, stirred at room temperature for 10 minutes, and then added to perfume oil A (25 g-see Table 2) at room temperature to form the oil phase. The latter was mixed with a solution of L-Lysine (2.53 g) in tap water (94.17 g). The reaction mixture was stirred with an Ultra Turrax at 24,000 rpm for 30 s to afford an emulsion. Ethylene diamine (0.12 g) and diethylene triamine (0.22 g) were dissolved in tap water (5 g) and this solution was added dropwise to the emulsion over the period of five minutes. The reaction mixture was stirred at 60° C. for 4 h to afford a white dispersion.

TABLE 2
Perfume oil A composition

| Chemical name | Amount (% wt) |
|---|---|
| Isopropyl myristate | 0.3 |
| (Z)-3-hexen-1-ol butyrate | 0.6 |
| Delta damascone | 1.0 |
| 2,4-Dimethyl-3-cyclohexene-1-carbaldehyde | 1.0 |
| Habanolide ® ¹⁾ | 3.0 |
| Hedione ® ²⁾ | 5.0 |
| Hexyl cinnamic aldehyde | 12.0 |
| Iso E Super ®³⁾ | 16.0 |
| Verdyl acetate | 24.0 |
| Lilial ®⁴⁾ | 37.0 |

¹⁾ Trademark from Firmenich; pentadecenolide, origin: Firmenich SA, Geneva, Switzerland
²⁾ Trademark from Firmenich; Methyl-cis-3-oxo-2-pentyl-1-cyclopentane acetate, origin: Firmenich SA, Geneva, Switzerland
³⁾Trademark from IFF; 7-acetyl, 1,2,3,4,5,6,7,8-octahydro-1,1,6,7-tetramethyl naphthalene
⁴⁾Trademark from Givaudan; 3-(4-tert-butylphenyl)-2-methylpropanal

15 g of the microcapsule slurry (obtained from Protocol 1 or Protocol 2) is diluted in 135 g of an alkaline buffer solution (pH 9) (for protocol 1) or of an acetic acid buffer at pH 4 (for protocol 2) and 4.5 mL of 0.3 molar barium nitrate solution is added. The mixture is stirred by anchor stirrer in a closed reactor at 250 rpm until the barium ions have sufficient time to interact with the anionic surface of the microcapsules.

(i) 4.5 mL of a 0.3 molar solution of sodium sulfate is added slowly by syringe pump over 60 minutes (75 μL/min) to initiate the nucleation of mineral material at the capsule surface by precipitating the barium cations with the sulfate anions followed by an additional 60 minutes of stirring.

(ii) Equal 7.5 mL volumes of 0.3 molar barium nitrate and 0.3 molar sodium sulfate solutions are then simultaneously slowly added by syringe pump over 60 minutes (125 μL/min each) followed by an hour of stirring to allow for further mineral precipitation.

(iii) Equal 30 mL volumes of 0.3 molar barium nitrate and 0.3 molar sodium sulfate solutions are then slowly added over 120 minutes (250 μL/min) simultaneously followed by 60 minutes of stirring to allow for further mineral precipitation. This process is repeated twice more to generate a robust mineral shell in this example. The additions could be repeated systematically to achieve the desired mineral shell thickness and properties.

TABLE 3
Mineralization Parameters for Nucleation and Growth of Barium
Sulfate Based Mineral Layer (capsule A1 and capsule A2)

| Parameter | Precursor 1 Addition | | Precursor 2 Addition | |
|---|---|---|---|---|
| Reactant | Ba(NO₃)₂ | | Na₂SO₄ | |
| Amount | 8.00 | g | 4.35 | g |
| Concentration | 0.3M | | 0.3M | |
| Volume | 102 | mL | 102 | mL |

| | | |
|---|---|---|
| pH | 9.0 (or 4.0) | 9.0 (or 4.0) |
| Addition Time (hours) | 1 | 1 |
| Temperature (° C.) | RT (22) | RT (22) |
| Mixing Speed (rpm) | 250 | 250 |

### Example 2

### Preparation of Biopolymer-Based Capsules According to the Invention (B)

Microcapsules B were prepared using a similar protocol 1 as described in Example 1 with a composition as reported in Table 1, except that the biomineralization of capsules was conducted using the precursors listed in Table 4 a strontium phosphate mineral

TABLE 4
Mineralization Parameters for Nucleation and Growth of
Strontium Phosphate Based Mineral Layer (Capsule B)

| Parameter | Precursor 1 Addition | | Precursor 2 Addition | |
|---|---|---|---|---|
| Reactant | Sr(NO₃)₂ | | Na₂HPO₄ | |
| Amount | 6.48 | g | 2.89 | g |
| Concentration | 0.3M | | 0.2M | |
| Volume | 102 | mL | 102 | mL |

| | | |
|---|---|---|
| pH | 9.0 | 9.0 |
| Addition Time (hours) | 1 | 1 |
| Temperature (° C.) | RT (22) | RT (22) |
| Mixing Speed (rpm) | 250 | 250 |

### Example 3

### Preparation of Biopolymer-Based Capsules According to the Invention (C)

Microcapsules C were prepared using a similar protocol 1 as described in Example 1 with a composition as reported in Table 1, except that the biomineralization of capsules was conducted using the precursors listed in Table 5 a magnesium carbonate mineral.

TABLE 5
Mineralization Parameters for Nucleation and Growth of
magnesium carbonate Based Mineral Layer (capsule C)

| Parameter | Precursor 1 Addition | | Precursor 2 Addition | |
|---|---|---|---|---|
| Reactant | MgCl₂ | | Na₂CO₃ | |
| Amount | 2.91 | g | 3.24 | g |
| Concentration | 0.3M | | 0.3M | |
| Volume | 102 | mL | 102 | mL |

| | | |
|---|---|---|
| pH | 9.0 | 9.0 |
| Addition Time (hours) | 1 | 1 |
| Temperature (° C.) | RT (22) | RT (22) |
| Mixing Speed (rpm) | 250 | 250 |

### Example 4

### Preparation of Biopolymer-Based Control Capsules (X)

Control microcapsules X were prepared using a similar protocol 1 as described in Example 1 with a composition as reported in Table 1, except that the control capsules are unmodified (i.e without mineralization).

### Example 5

### Capsules Characterization and Deposition Results

### Microscopy of Capsules:

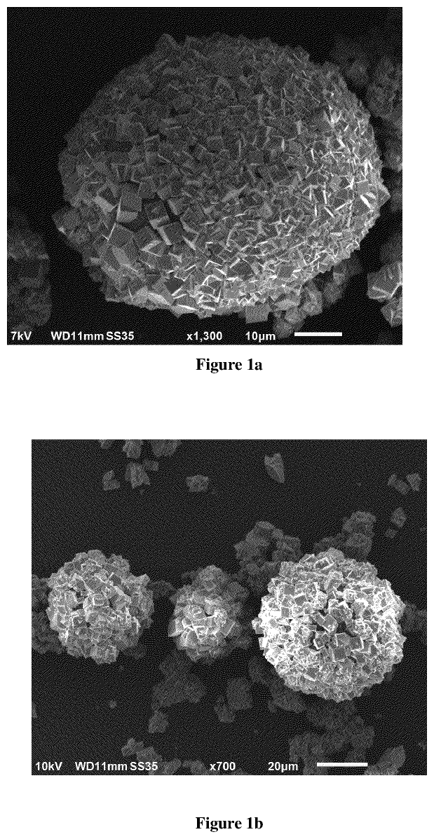

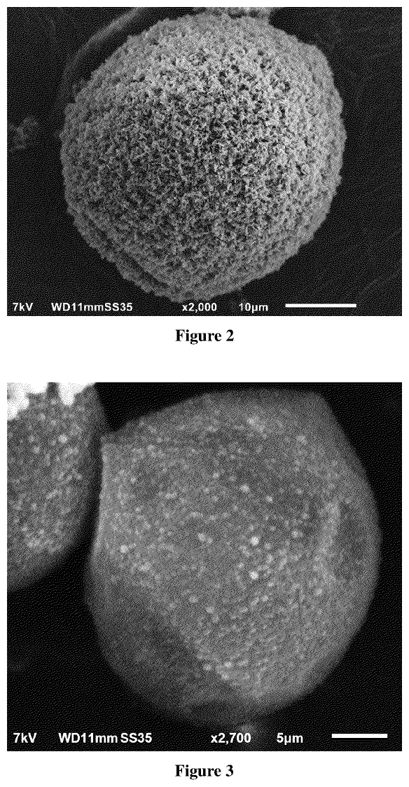

To image the microcapsules, dilute capsule slurries were dried onto carbon tape, which was adhered to aluminium stubs and then sputter coated with a gold/palladium plasma. The stubs were placed into a scanning electron microscope (JEOL 6010 PLUS LA) for analysis. Images of Capsules A1, Capsules A2, Capsules B, and Capsules C are shown respectively in FIG. 1a, FIG. 1b, FIG. 2, and FIG. 3 to illustrate that stable, robust, rough mineralized microcapsules can be generated by growing a crystalline mineral coating onto smooth polyurea microcapsule scaffolds.

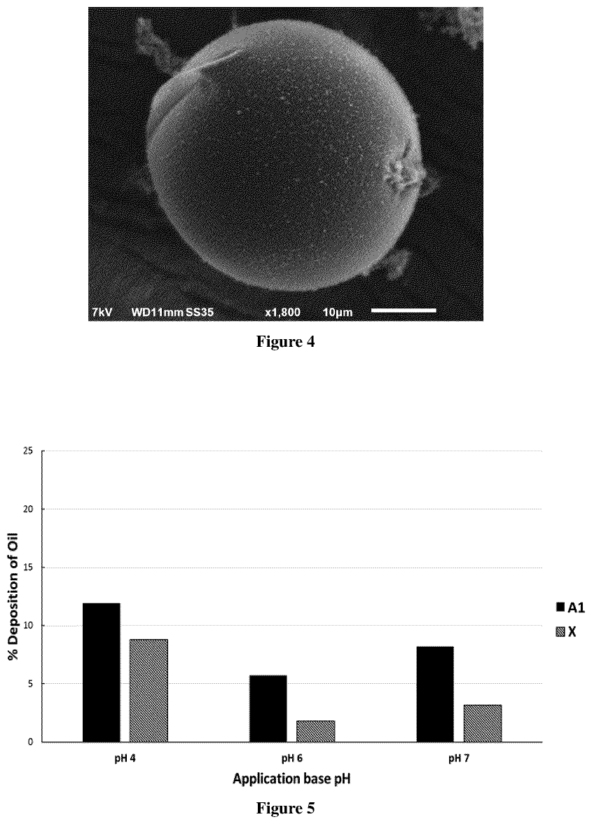

By contrast, comparative microcapsules X have a smooth, unmodified surface (FIG. 4).

### Deposition Testing on Hair:

For the quantification of deposition onto hair, the following procedure was used. A 500 mg mini hair swatch was wet with 40 mL of tap water (37-39° C.) aimed at the mount with a 140 mL syringe. The excess water was gently squeezed out once and 0.1 mL of a model surfactant mixture containing microcapsules loaded with a UV tracer (Uvinul A Plus) was applied with a 100 μL positive displacement pipet. The surfactant mixture was distributed with 10 horizontal and 10 vertical passes. The swatch was then rinsed with 100 mL of tap water (37-39° C.) with 50 mL applied to each side of the swatch aimed at the mount. The excess water was gently squeezed out and the hair swatch was then cut into a pre-weighed 20 mL scintillation vial. This process was repeated 2 more times and then the vials containing the cut hair were dried in a vacuum oven @ 50-60° C. (100 Torr) for at least 5 hours. After the drying process, the vials were again weighed to determine the mass of the hair in the vials. Controls were also prepared by adding 0.1 mL of the model surfactant mixture containing capsules to an empty vial. 4 mL of 200-proof ethanol were then added to each vial and they were subjected to 60 minutes of sonication. After sonication, the samples were filtered through a 0.45 μm PTFE filter and analyzed with a HPLC using a UV detector. To determine the percent deposition of microcapsules from a model surfactant mixture, the amount of Uvinul extracted from the hair samples was compared to the amount of Uvinul extracted from the control samples.

TABLE 6
Model surfactant mixture

| | Active | |
|---|---|---|
| Ingredients | Amount (% wt) | Function |
| Sodium Laurel Ether Sulfate | 12 | Anionic Surfactant |
| (SLES) | | |
| Cocamidopropyl Betaine | 3 | Amphoteric Surfactant |
| (CAPB) | | |
| Salcare ® SC 60 Polymer¹⁾ | 0.5 | Deposition Aid |
| Water | 84 | Solvent |
| Microcapsule Slurry | 0.5 | Fragrance |
| (Equivalent Oil) | | |

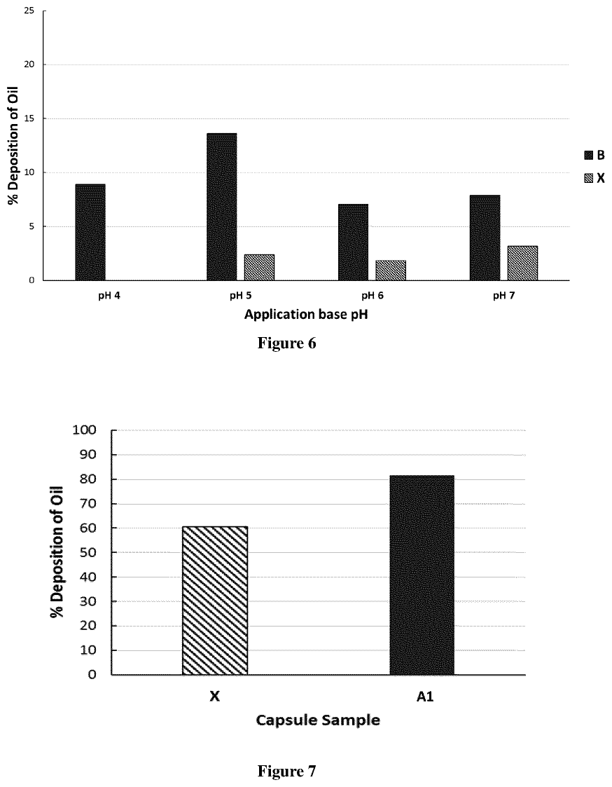

¹⁾acrylamidopropyltrimonium chloride/acrylamide copolymer; origin BASF Deposition onto hair swatches was measured from this simplified model surfactant mixture which is meant to be representative of personal cleansing formulations such as shampoo or shower gel. Results are shown in FIG. 5 for Capsules A1 and FIG. 6 for Capsules B.

The data illustrated in FIG. 5 demonstrate that the addition of a barium sulfate mineral layer to an anionic biopolymer-stabilized capsule increases the deposition onto hair swatches significantly from 1.8% for the control capsules X to more than 5.8% for the mineralized Capsules A1 at standard formulation pH, and can reach deposition percentage as high as 12% in pH 4. The capsules according to the invention are boosting deposition up to 3 times better than prior art capsules and benefits are demonstrable from pH 4 to pH 7.

The data illustrated in FIG. 6 demonstrate that the addition of a strontium phosphate mineral layer to an anionic biopolymer-stabilized capsule increases the deposition onto hair swatches significantly from 2.4% for the control capsules X to more than 13.6% for the mineralized Capsules B at standard formulation pH. The capsules according to the invention are boosting deposition up to 5.6 times better than prior art capsules with benefits demonstrable from pH 5 to pH 7.

### Deposition Testing on Fabric:

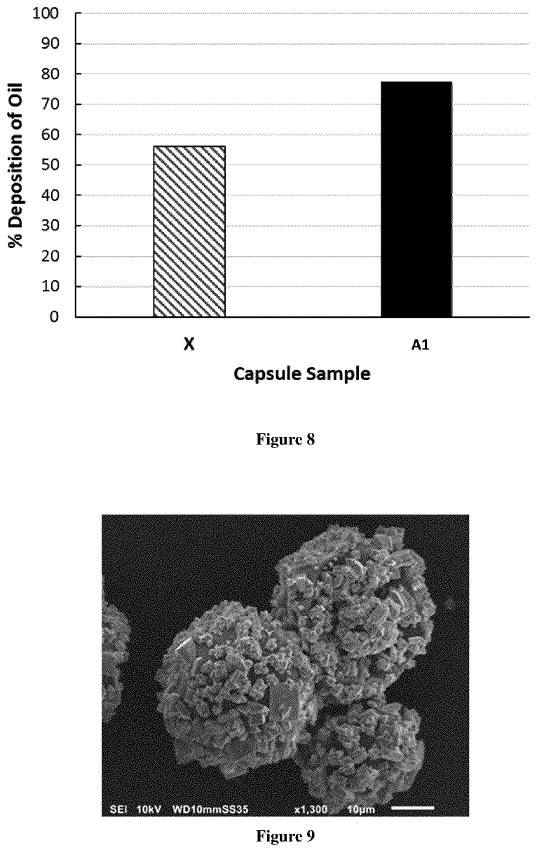

For the quantitative deposition of microcapsules onto fabric, a 1.0 g cotton towel swatch was subjected to a miniaturized laundry simulation process for quick screening. A 50 mL centrifuge tube was used as the model laundry vessel and an IKA high-speed (fixed) vortexer was used to simulate the washing machine action. 30 mL of tap water (room temperature) was placed into the centrifuge tube, and a positive displacement pipette was used to add 100 μL of laundry care base containing microcapsules loaded with a UV tracer. The 1.0 g swatch of white, decontaminated cotton towel was placed into the centrifuge tube, which was then capped and placed onto the vortexer for 30 seconds to thoroughly mix the contents. For testing deposition from fabric softener, the liquid was then poured out and the towel swatch was lightly wrung out by gently rolling a pipette across the surface to push out the excess water without squeezing the capsules, and the swatch was line dried overnight. For testing from detergent, the swatches were submerged in another 30 mL of clean tap water and subjected to another 30 seconds on the vortexer to simulate the rinse cycle before emptying the water and wringing the towels with the pipette to remove excess water and line drying overnight. The dry swatches were then submerged in 10 mL of ethanol (HPLC grade, 200 proof) and sonicated for 1 hour in a sonicating bath to rupture the capsules and extract the deposited oil containing the Uvinul A+UV tracer. Simultaneously, controls of 100 μL of the fabric softener base containing the capsules were placed in scintillation vials with 4 mL of ethanol and extracted via sonication to determine the total oil content loaded into the miniature laundry simulators. The ethanol containing the extracted UV tracer from the oil was run through an HPLC (Luna C8 column) with a UV-Vis detector to back calculate the oil content extracted from each sample. The oil content values of the controls were compared to the oil content values found on the fabric swatches (accounting for dilutions) to determine the percentage of total oil deposited on the fabric. The results of the quantitative deposition assessment from fabric softener are shown in FIG. 7. Results from detergent are shown in FIG. 8.

The data illustrated in FIG. 7 demonstrate that the addition of a barium sulfate mineral layer to an anionic biopolymer-stabilized capsule increases the deposition onto fabric from a fabric softener base significantly from 61% for the control capsules X to 82% for the mineralized Capsules A1, representing a 31% increase in deposited oil.

The data illustrated in FIG. 8 demonstrate that the addition of a barium sulfate mineral layer to an anionic biopolymer-stabilized capsule increases the deposition onto fabric from a detergent base significantly from 56% for the control capsules X to 77% for the mineralized Capsules A1, representing a 37% increase in deposited oil.

### Example 6

### Stability in a Low pH Surfactant Composition

FIG. 9 represents scanning electron micrographs of mineralized microcapsules according to the invention (Capsules A1) that have been incubated for one month in the fabric softener composition according to Table 1.

### Example 7

### Fabric Softener Composition

Microcapsules of the present invention are dispersed in a fabric softener composition to obtain a concentration of encapsulated perfume oil at 0.116%.

TABLE 7
Fabric Conditioner composition

| Product | Wt % |
|---|---|
| Stepantex VL 90A | 8.88 |
| Calcium Chloride Sol. 10% | 0.36 |
| Proxel GXL | 0.04 |
| Perfume | 1.00 |
| Water | 89.72 |
| TOTAL | 100 |

### Example 8

### Liquid Detergent Composition

Microcapsules of the present invention are dispersed in a liquid detergent base to obtain a concentration of encapsulated perfume oil at 0.22%.

TABLE 8
Liquid detergent composition

| | Concentration |
|---|---|
| Ingredients | [wt %] |
| Sodium C14-17 Alkyl Sec Sulfonate¹⁾ | 7 |
| Fatty acids, C12-18 and C18-unsaturated²⁾ | 7.5 |
| C12/14 fatty alcohol polyglycol ether with 7 | 17 |
| mol EO³⁾ | |
| Triethanolamine | 7.5 |
| Propylene Glycol | 11 |
| Citric acid | 6.5 |
| Potassium Hydroxyde | 9.5 |
| Protease | 0.2 |
| Amylase | 0.2 |
| Mannanase | 0.2 |
| Acrylates/Steareth-20 Methacrylate | 6 |
| structuring Crosspolymer⁴⁾ | |
| Deionized Water | 27.4 |

¹⁾Hostapur ® SAS 60; Origin: Clariant
²⁾Edenor ® K 12-18; Origin: Cognis
³⁾Genapol ® LA 070; Origin: Clariant
⁴⁾Aculyn ® 88; Origin: Dow Chemical

### Example 9

### Unit Dose Formulation

A sufficient amount of exemplified microcapsules is weighed and mixed in a unit dose formulation to add the equivalent of 0.2% perfume.

The unit dose formulation can be contained in a PVOH (polyvinyl alcohol) film.

TABLE 9
Unit dose composition

| | Concentration |
|---|---|
| Ingredients | [wt %] |
| C12-C14 alkyl poly ethoxylate | 15 |
| C12-C14 alkyl poly ethoxylate sulfate | 9.5 |
| Mono Ethanol Amine salt | |
| Linear Alkylbenzene sulfonic acid | 17 |
| Citric Acid | 0.5 |
| C12-C18 Fatty Acid | 17 |
| Enzymes | 1.2 |
| Fluorescent brightener | 0.3 |
| 1,2 propanediol | 12 |
| Glycerol | 9 |
| Sodium Hydroxide | 1 |
| Mono Ethanol Amine | 6 |
| PDMS | 2.5 |
| Potassium sulphite | 0.2 |
| water | 8.8 |
| Total | 100 |

### Example 10

### Rinse-Off Conditioner

Microcapsules of the present invention are dispersed in a rinse-off conditioner base to obtain a concentration of encapsulated perfume oil at 0.5%.

TABLE 10
Rinse-off conditioner composition

| | | Concentration |
|---|---|---|
| | Ingredients | [wt %] |
| A | Water deionized | 81.8 |
| | Behentrimonium Chloride ¹⁾ | 2.5 |
| | Hydroxyethylcellulose ²⁾ | 1.5 |
| B | Cetearyl Alcohol ³⁾ | 4 |
| | Glyceryl Stearate (and) PEG-100 Stearate ⁴⁾ | 2 |
| | Behentrimonium Methosulfate (and) Cetyl alcohol | 4 |
| | (and) Butylene Glycol ⁵⁾ | |
| | Ethoxy (20) Stearyl Alcohol ⁶⁾ | 1 |
| C | Amodimethicone (and) Trideceth-12 (and) | 3 |
| | Cetrimonium Chloride ⁷⁾ | |
| | Chlorhexidine Digluconate ⁸⁾ 20% | 0.2 |
| | aqueous solution | |
| D | Citric acid 10% aqueous sol. till pH 3.5-4 | q.s. |
| | TOTAL: | 100 |

¹⁾ Genamin KDM P, Clariant
²⁾ Tylose H10 Y G4, Shin Etsu
³⁾ Lanette O, BASF
⁴⁾ Arlacel 165-FP-MBAL-PA-(RB), Croda
⁵⁾ Incroquat Behenyl TMS-50-MBAL-PA-(MH) HA4112, Croda
⁶⁾ SP Brij S20 MBAL-PA(RB), Croda
⁷⁾ Xiameter DC MEM-0949 Emulsion, Dow Corning
⁸⁾ Alfa Aesar

### Example 11

### Shampoo Composition

Microcapsules of the present invention are weighed and mixed in a shampoo composition to add the equivalent of 0.2% perfume.

TABLE 11
Shampoo composition

| | | Concentration |
|---|---|---|
| | Ingredients | [wt %] |
| A | Water deionized | 44.4 |
| | Polyquaternium-10 ¹⁾ | 0.3 |
| | Glycerin 85% ²⁾ | 1 |
| | DMDM Hydantoin ³⁾ | 0.2 |
| B | Sodium Laureth Sulfate ⁴⁾ | 28 |
| | Cocamidopropyl Betaine ⁵⁾ | 3.2 |
| | Disodium Cocoamphodiacetate ⁶⁾ | 4 |
| | Ethoxy (20) Stearyl Alcohol ⁶⁾ | 1 |
| C | Sodium Laureth Sulfate ⁴⁾ | 3 |
| | Glyceryl Laureate ⁷⁾ | 0.2 |
| D | Water deionized | 1 |
| | Sodium Methylparaben ⁸⁾ | 0.1 |
| E | Sodium Chloride 10% aqueous sol. | 15 |
| | Citric acid 10% aqueous sol. till pH 5.5-6 | q.s. |
| | Perfume | 0.5 |
| | TOTAL: | 100 |

¹⁾ Ucare Polymer JR-400, Noveon
²⁾ Schweizerhall
³⁾ Glydant, Lonza
⁴⁾ Texapon NSO IS, Cognis
⁵⁾ Tego Betain F 50, Evonik
⁶⁾ Amphotensid GB 2009, Zschimmer & Schwarz
⁷⁾ Monomuls 90 L-12, Gruenau
⁸⁾ Nipagin Monosodium, NIPA

### Example 12

### Antiperspirant Roll-on Emulsion Composition

Microcapsules of the present invention are weighed and mixed in antiperspirant roll-on emulsion composition to add the equivalent of 0.2% perfume.

TABLE 12
Antiperspirant roll-on emulsion composition

| Ingredient | Amount (wt %) |
|---|---|
| Steareth-2¹⁾ (Part A) | 3.25 |
| Steareth-21²⁾ (Part A) | 0.75 |
| PPG-15 Stearyl Ether³⁾ (Part A) | 4 |
| WATER deionised (Part B) | 51 |
| Aluminum Chlorohydrate 50% | 40 |
| aqueous solution⁴⁾ (Part C) | |
| Fragrance (Part D) | 1 |

¹⁾BRIJ 72; origin: ICI
²⁾BRIJ 721; origin: ICI
³⁾ARLAMOL E; origin: UNIQEMA-CRODA
⁴⁾LOCRON L; origin: CLARIAN

Part A and B are heated separately to 75° C.; Part A is added to Part B under stirring and the mixture is homogenized for 10 min. Then, the mixture is cooled under stirring; and Part C is slowly added when the mixture reached 45° C. and Part D when the mixture reached at 35° C. while stirring. Then the mixture is cooled to room temperature.

### Example 13

### Deodorant Spray Composition

Microcapsules of the present invention are weighed and mixed in antiperspirant roll-on emulsion composition to add the equivalent of 0.2% perfume.

TABLE 13
Deodorant spray composition

| Ingredient | Amount (wt %) |
|---|---|
| Ethanol 95% | 90.65 |
| Triclosan¹⁾ | 0.26 |
| Isopropyl miristate | 9.09 |

¹⁾Irgasan DP 300; trademark and origin: BASF

All the ingredients according to the sequence of Table 11 are mixed and dissolved. Then the aerosol cans are filled, crimp and the propellant is added (Aerosol filling: 40% active solution 60% Propane/Butane 2.5 bar).

### Example 14

### Shower-Gel Composition

Microcapsules of the present invention are weighed and mixed in the following composition to add the equivalent of 0.2% perfume.

TABLE 14
Shower gel composition

| | Amount | |
|---|---|---|
| Ingredients | (% wt) | Function |
| WATER deionized | 49.350 | Solvent |
| Tetrasodium EDTA ¹⁾ | 0.050 | Chelating agent |
| Acrylates Copolymer²⁾ | 6.000 | Thickener |
| Sodium C12-C15 Pareth Sulfate ³⁾ | 35.000 | Surfactant |
| Sodium Hydroxide 20% aqueous solution | 1.000 | pH adjuster |
| Cocamidopropyl Betaine⁴⁾ | 8.000 | Surfactant |
| Methylchloroisothiazolinone and | 0.100 | Preservative |
| Methylisothiazolinone⁵⁾ | | |
| Citric Acid (40%) | 0.500 | pH adjuster |

6) EDETA B POWDER; trademark and origin: BASF
7) CARBOPOL AQUA SF-1 POLYMER; trademark and origin: NOVEON
8) ZETESOL AO 328 U; trademark and origin: ZSCHIMMER & SCHWARZ
9) TEGO-BETAIN F 50; trademark and origin: GOLDSCHMIDT
10) KATHON CG; trademark and origin: ROHM & HASS

### Example 15

### Toothpaste Formulation

A sufficient amount of a microcapsule slurry M (prepared according to the protocol 1 disclosed in example 1 except that a menthol flavor is encapsulated) is weighed and mixed in the following composition to add the equivalent of 0.2% flavor.

TABLE 15
Toothpaste formulation

| Ingredients | Amount (% wt) |
|---|---|
| Polyethylene glycol 400 | 2.0% |
| Xanthan Gum | 0.6% |
| Sorbitol 70% Solution | 50% |
| Sodium Fluoride | 0.220% |
| Sodium Benzoate | 0.2% |
| Water | 15.230% |
| Hydrated Silica¹⁾ | 22.0% |
| Hydrated Silica²⁾ | 7.0% |
| Titanium Dioxide CI77891 | 0.5% |
| Sodium Lauryl Sulfate | 1.250% |
| Flavor | 1.2% |
| TOTAL | 100% |

¹⁾Tixosil 73; trademark and origin:
²⁾Tixosil 43; trademark and origin:

### Example 16

### Dicalcium Phosphate Based Toothpaste Formulation

A sufficient amount of a microcapsule slurry M (prepared according to the protocol 1 disclosed in example 1 except that a menthol flavor is encapsulated) is weighed and mixed in the following composition to add the equivalent of 0.2% flavor.

TABLE 16
Toothpaste formulation

| Ingredients | Amount (% wt) |
|---|---|
| Sodium carboxymethyl cellulose | 1.2% |
| Flavor | 1.2% |
| DI/Purified Water | Q.S to Final Wt. |
| Sodium Lauryl Sulfate | 1.3% |
| Glycerine | 20.0% |
| Sodium Saccharin | 0.2% |
| Dicalcium phosphate dihydrate | 36.0% |
| Methylparaben | 0.2% |
| Silica¹⁾ | 3.0% |
| TOTAL | 100% |

¹⁾Aerosil ®200; trademark and origin:

### Example 17

### Mouthwash Alcohol Free Formulation

A sufficient amount of a microcapsule slurry M (prepared according to the protocol 1 disclosed in example 1 except that a menthol flavor is encapsulated) is weighed and mixed in the following composition to add the equivalent of 0.2% flavor.

TABLE 17
Mouthwash formulation

| Ingredients | Amount (% wt) |
|---|---|
| Propylene Glycol | 10% |
| Flavor | 0.240% |
| DI/Purified Water | Q.S to Final Wt. |
| Poloxamer 407 NF | 0.240% |
| Sodium Lauryl Sulfate | 0.040% |
| Sorbitol 70% Solution | 10.0% |
| Sodium Saccharin | 0.030% |
| Glycerine | 3.0% |
| Sodium Benzoate | 0.100% |
| Sucralose | 0.020% |
| Benzoic Acid | 0.050% |
| TOTAL | 100% |

### Example 18

### Mouthwash Formulation

A sufficient amount of a microcapsule slurry M (prepared according to the protocol 1 disclosed in example 1 except that a menthol flavor is encapsulated) is weighed and mixed in the following composition to add the equivalent of 0.2% flavor.

TABLE 18
Mouthwash formulation

| Ingredients | Amount (% wt) |
|---|---|
| Ethyl Alcohol 190 Proof | 15.0% |
| Flavor | 0.240% |
| DI/Purified Water | Q.S to Final Wt. |
| Poloxamer 407 NF | 0.240% |
| Sodium Lauryl Sulfate | 0.040% |
| Sorbitol 70% Solution | 10.0% |
| Sodium Saccharin | 0.030% |
| Glycerine | 3.0% |
| Sodium Benzoate | 0.100% |
| Sucralose | 0.020% |
| Benzoic Acid | 0.050% |
| TOTAL | 100% |

1. A mineralized core-shell microcapsule comprising:
a) a core comprising a hydrophobic material;
b) a shell having a terminating charged functional surface; and
c) a mineral layer on the terminating charged functional surface,
wherein the mineral layer comprises at least one salt selected from the group consisting of barium salt, strontium salt, magnesium salt, and mixtures thereof.

2. The mineralized core-shell microcapsule according to claim 1, wherein the salt is selected from the group consisting of carbonate, strontium phosphate, magnesium barium sulfate, strontium sulfate, strontium phosphate, magnesium carbonate and mixtures thereof.

3. The microcapsule according to claim 1, wherein the terminating surface is an anionic surface and wherein the microcapsule comprises a polyelectrolyte scaffolding between the anionic surface and the mineral layer, said polyelectrolyte scaffolding including at least one cationic polyelectrolyte layer and at least one anionic polyelectrolyte layer with the proviso that the terminating layer is an anionic polyelectrolyte layer.

4. The microcapsule according to claim 1, wherein the shell is a polymeric shell comprising a material selected from the group consisting of polyurea, polyurethane, polyamide, polyhydroxyalkanoates, polyacrylate, polyesters, polyaminoesters, polyepoxides, polysiloxane, polycarbonate, polysulfonamide, urea formaldehyde, melamine formaldehyde resin, melamine formaldehyde resin cross-linked with polyisocyanate or aromatic polyols, melamine urea resin, melamine glyoxal resin, gelatin/gum arabic shell wall, and mixtures thereof.

5. The microcapsule according to claim 1, wherein the core is an oil-based core.

6. A process for preparing a mineralized core-shell microcapsule the mineralized core-shell microcapsule of claim 1, the process comprising the steps of:
(i) preparing a core-shell microcapsule slurry comprising microcapsules having a terminating charged functional surface;
(ii) adsorbing at least one mineral precursor on the charged surface; and
(iii) applying conditions suitable to induce crystal growth of the mineral on the charged surface to form a mineral layer,
wherein the mineral precursor is adsorbed on the charged surface by incubating the core-shell microcapsule slurry obtained in step (i) in at least one mineral precursor solution, wherein the mineral precursor solution is selected from the group consisting of barium salt solution, strontium salt solution, magnesium salt solution, phosphate-based salt solution, sulfate-based salt solution, carbonate-based salt solution and mixtures thereof.

7. The process according to claim 6, wherein the microcapsule core-shell slurry in step (i) is formed by interfacial polymerisation or by precipitation in the presence of a charged emulsifier.

8. The process according to claim 7, wherein the charged emulsifier is an anionic emulsifier and forms an anionic surface when the interfacial polymerization or precipitation is completed in step (i).

9. The process according to claim 7, wherein the charged emulsifier is a cationic emulsifier that forms a cationic surface when the interfacial polymerization is completed, and wherein step (i) further comprises a step of coating at least one anionic polyelectrolyte layer on the cationic surface to form core-shell microcapsule having a terminating anionic functional surface.

10. A consumer product comprising the mineralized core-shell microcapsule of claim 1.

11. The consumer product according to claim 10, wherein the consumer product is in the form of a perfumed consumer product.

12. The consumer product according to claim 10, wherein the consumer product is in the form of a flavoured consumer product.

13. The microcapsule according to claim 1, wherein the core is an oil-based core comprising a perfume.

14. The consumer product according to claim 10, wherein the consumer product is in the form of a laundry care product, a home care product, a body care product, a hair care product, a skin care product, an air care product, or a hygiene product.
